(19) United States
(12) Patent Application Publication
(10) Pub. No.: US 2024/0207403 A1
(43) Pub. Date: Jun. 27, 2024

(54) PERSONALIZED IMMUNOTHERAPY USING INTESTINAL METABOLITES

(71) Applicant: Evolve Biosystems, Inc., Davis, CA (US)

(72) Inventors: Bethany HENRICK, Davis, CA (US); Petter BRODIN, Davis, CA (US)

(21) Appl. No.: 18/557,792

(22) PCT Filed: Apr. 29, 2022
(86) PCT No.: PCT/US2022/027154

§ 371 Date: Oct. 27, 2023

Related U.S. Application Data
(60) Provisional application No. 63/181,816, filed on Apr. 29, 2021.

(51) Int. Cl. A61K 39/00; A61K 31/19; A61K 31/405; A61K 35/24; A61K 35/745; A61K 35/747; A61K 38/18; A61K 38/20; A61K 38/21; A61P 37/06; C12N 1/20; C12N 5/0783

(52) U.S. Cl. CPC A61K 39/4621; A61K 31/19; A61K 31/405; A61K 35/24; A61K 35/745; A61K 35/747; A61K 38/1841; A61K 38/20; A61K 38/2013; A61K 38/2066; A61K 38/215; A61K 38/217; A61K 39/4611; A61P 37/06; C12N 1/20; C12N 5/0636

(57) ABSTRACT

Disclosed is a T cell polarizing composition comprising a plurality of metabolites selected from metabolites enriched in non-dysbiotic feces, preferably including one or more of acetate, lactate, indole 3 lactate, 3-(4-hydroxyphenyl)lactate, or phenyllactate, bile acids (cholate, chenodeoxycholate, cholate sulfate), or IFNbeta. Further disclosed is a method of producing the T cell polarizing composition comprising mixing interferon beta (IFNbeta), acetate, lactate, and/or ILA.

### BACKGROUND

The ability to change the infant gut microbiome using certain bacteria and certain mammalian milk oligosaccharides (MMO) has previously been described. In particular B. infantis has been shown to take a predominant role in the intestinal microbiome of infants fed a diet containing human milk. This resulted in biochemical, immune and metabolic changes in the gut (Frese 2017, Henrick 2019, WO 2018/006080, WO2019/055718B).

While immune changes have been associated with gut composition, a new fecal marker interferon beta (IFNβ) is emerging in relation to the infant gut microbiome and development of the immune system.

Certain immune cells, such as dendritic cells and gut associated lymphoid tissue act at the interface between the host intestinal epithelium in the lamina propria and the lumen of the gut where the bacteria reside. This interaction between host and the intestinal lumen is a mechanism that is largely understood by persons skilled in the arts of immunology.

The role of global changes in enteric cytokines and other immune factors at the level of the gut are less understood in the context of health or disease.

Infant immune system has maximum flexibility in the first 100 days and is an important mechanism for an individual to develop oral tolerance to a wide range of antigens in the environment.

IFNβ is used as an intravenous therapy for autoimmune diseases like MS and in acute viral infections to stop cytokine storms.

Early gut microbiome dysbiosis, described as an overabundance of proteobacteria (Shin et al., 2015) and loss of ecosystem function [Duar et al, 2020] is associated with both acute and chronic immune dysregulation, leading to common conditions such as colic (Rhoads et al., 2018) atopic wheeze and allergy (Arrieta et al., 2015, 2017; Laforest-Lapointe and Arrieta, 2017) and less common, but serious immune-mediated disorders such as type-1 diabetes (Vatanen et al., 2016) and Crohn's disease (Hviid et al., 2011). Immune development has been poorly understood in humans due to the difficulty in obtaining samples from infants. Recent developments in systems immunology enable profiling of immune development at the systems level and unraveling immune cell regulatory relationships (Davis and Brodin, 2018). Advances in sample processing mean that small volume samples available from newborn infants are no longer prohibitive and as little as 100 μL of whole blood is sufficient for systems-level immunomonitoring and postnatal adaptation by the newborn immune system to environmental exposures is being elucidated (Olin et al., 2018).

Dysbiosis of the infant gut microbiome is common in modern societies and a likely contributing factor to the increased incidences of immune-mediated disorders (Dominguez-Bello et al., 2019; Mohammadkhah et al., 2018; Sonnenburg and Sonnenburg, 2019). Therefore, there is great interest in identifying microbial factors that can support healthier immune system imprinting and prevent cases of allergy, autoimmunity and possibly other conditions involving the immune system (Renz and Skevaki, 2020). Loss of Bifidobacterium early in life has been associated with increased risk of developing autoimmunity as seen in a birth cohort in Finland (Vatanen et al., 2016) and atopic wheeze in another cohort in rural Ecuador (Arrieta et al., 2017). Moreover, observational studies have identified a link between the loss of Bifidobacterium in infants and enteric inflammation early in life, but the mechanisms involved are elusive (Henrick et al., 2019; Rhoads et al., 2018).

Human breast milk contains abundant human milk oligosaccharides (HMOs) that are not digestible by humans who lack necessary glycosidases. Instead, the maternal energy spent to create such complex sugars is justified by providing selective nutritional advantage to “beneficial” microbes specialized in metabolizing HMOs. Bifidobacterium longum subspecies infantis (B. infantis) is one such strain adapted to metabolizing HMOs. B. infantis is commonly found in breastfed infants in countries where incidence of immune-mediated disorders is low, such as Bangladesh (Huda et al., 2014), and Malawi (Grzekowiak et al., 2012), but rarely in Europe (Abrahamsson et al., 2014; Avershina et al., 2014; Jost et al., 2012; Roos et al., 2013) and North America (Azad et al., 2013; Casaburi et al., 2021; Lewis et al., 2015). Introducing B. infantis has been successfully accomplished, using strains such as B. infantis EVC001 which is able to stably and persistently colonize and dominate the intestinal microbiome of breastfed infants (Frese et al., 2017), leading to reduced fecal calprotectin, a marker of intestinal inflammation (Henrick et al., 2019).

### SUMMARY OF INVENTION

This invention includes compositions and methods that stimulate polarization of T cells towards enhanced T regulatory and Helper T cell profiles based on specific intestinal signals.

T cell polarizing compositions may comprise a plurality of metabolites selected from metabolites enriched in non-dysbiotic feces, preferably including, but not limited to one or more of acetate, lactate, indole 3 lactate, 3-(4-hydroxyphenyl)lactate, phenyllactate, bile acids (cholate, chenodeoxycholate, cholate sulfate), or cytokines that may include one or more of IFNβ, IL-2, IL-4, IL-5, IL-6, IL-8, IL-10, IL-13, IL-12p70, IL-17A, IL-21, IL-22, IL-27, IL-31, IL-33, MIP-3a, TNFα, TGFβ, IL-1b, and/or IFNγ. The non-dysbiotic feces or the metabolic profile of a non-dysbiotic infant's feces may be defined as a high abundance of human milk utilization genes in a potential donor feces, high lactate dehydrogenase 4 (LDHIV) expression, high abundance of at least greater than 50%, at least 60%, at least 70%, or at least 80% of Bifidobacterium species, or more specifically B. infantis, measurement of ILA, acetate or lactate and/or pH less than 5.5 or less than 5.1.

In some embodiments, the T cell polarizing composition may be used to stimulate a T cell preparation in vitro and/or be applied in vivo. Compositions include select combinations of gut microbiome metabolites and host signals. Such compositions are delivered ex vivo to polarize naïve T cells in a particular direction. A polarizing T cell composition is a made by one or more of the following methods that include sterile fecal water, a cell culture supernatant or spent media, an artificial stool, one or more purified bacteria and/or metabolites that may be enriched or purified from bacterial culture or other sources. The bacterial culture comprises at least HMO genes associated with metabolizing and consuming HMO, a preferable and representative bacterial species, such as B. infantis. A T cell polarizing composition may be a dried powder, but may also be a liquid, gel or paste that is formulated for delivery and administration to a patient in need in vivo or ex vivo as part of a treatment regime.

Compositions produced and used in methods described herein may have stems cells or naïve T cells harvested from individuals in need of additional regulatory T cells. Immunotherapy may be used on stem cells or naïve T cells from individuals with autoimmune disease, allergy, or cancer.

This invention provides a T cell preparation comprising cultured human T regulatory cells and helper T cells polarized from a purified naïve T cell population; and an activity profile comprising desired frequency or ratio of T regulatory and T helper cell types suitable for delivery to a patient in need.

In some embodiments, a T cell population is altered ex vivo, wherein said method comprises i) collecting blood; ii) isolating naïve T cells; iii) exposing naïve T cells to a polarizing composition; iv) recovering polarized T cells; and v) administering a composition of the T cell preparation.

In some embodiments, stem cells obtained from a subject are cultured under conditions whereby at least some of the cells differentiate into T cells, and wherein any polarizing composition is applied to the T cells and polarized T cells are recovered after polarization. Any polarizing composition of this invention is applied to a population of naïve T cells separated from other T cells, or may be applied to a mixed population of T cells separated from other white blood cells. In some embodiments, the subject in need of immunotherapy is the source of the naïve T cells and the recovered polarized T cells are reintroduced into the same subject. Alternatively, naïve T cells may be sourced from a compatible donor.

A desired activity profile comprises a frequency or ratio of specific types of T cells, such as regulatory T cells, helper T cells (Type 1 or Type 2) and/or naïve T cells that may be defined as frequency i.e. percentage of final CD+ T cell population measured. In the final T cell, preparation, the frequency of residual naïve T cells is less than 1%, less than 5%, or less than 10% Purified naïve T cells frequency may be determined by expression CD3+CD4+CD45RA+ and/or lack of expression for CD8, CD14, CD19, or CD56. Purified naïve T cells, are taken to be a preparation of whole blood that is processed to enrich for naïve T cells and/or deplete other cell types. A composition of this invention may have the cell frequency of at least 1%, at least 2%, at least 5%, at least 10%, at least 20%, at least 40%, or at least 80% of the total CD4+ T cell population in the T cell preparation are regulatory T cells expressing FOXP3+. The frequency of at least 10%, at least 15%, at least 25%, at least 30%, at least 40%, at least 50%, at least 60%, at least 70%, and at least 80% of the total CD4+ T cell population in the T cell preparation are T helper Type I (Th1) cells. The frequency of less than 50%, less than 40%, less than 30%, less than 20%, less than 10%, less than 5%, less than 1% of the T cells in the T cell preparation are T helper Type 2 cells (Th2). The frequency of less than 50%, less than 40%, less than 30%, less than 20%, less than 10%, less than 5%, or less than 1% of the T cells in the T cell preparation are T helper type 17 (Th17) cells. In some embodiments, a composition of this invention is a composition wherein galectin-1 expression is upregulated in T cell preparation compared to naïve T cells and/or an IFNβ is present.

In some embodiments, the ratio of FoxP3+CD4+ T cells to Th2 or Th17 CD4+ T cells may be greater than 1:1, 2:1, 4:1, 8:1, 16:1, 32:1, 64:1, 128:1, 256:1, 512:1, 1024:1. In others, the ratio of Th1 to Th2 CD4+ T cells may be greater than 1:1, 2:1, 4:1, 8:1, 16:1, 32:1, 64:1, 128:1.

The cultured human T regulatory and helper T cells in a T cell preparation are at least 10 cultured cells per unit dose, at least 100 cultured cells per unit dose, at least 1000 cultured cells per unit dose, at least 10,000 cultured cells per unit dose, at least 100,000 cultured cells per unit dose, at least 1 million cultured cells per unit dose, or at least 1 billion cultured cells per unit dose. Preferably, the cultured human T regulatory and helper T cells in a T cell preparation are in a dose range of 10¹-10⁹ cells/ml, 10¹-10³ cells/ml, 10²-10⁴ cells/ml, or 10⁴-10⁷ cells/ml.

This invention provides a method of increasing desirable and/or depleting undesirable circulating T cells, wherein the method comprises using any polarization composition to alter T cell populations. The polarizing composition may be administered to treat a subject in need of increasing circulating regulatory T cells to 5-10% of all CD4+ T cells. For instance, the administration of the polarizing composition may inhibit Th17 cell expansion in vivo by at least 10%, at least 30%, or at least 50% relative to the Th17 levels prior to administration of the polarizing composition.

Compositions and methods described herein may be used to enhance immune expansion and/or reduce intestinal inflammation. Particularly, the compositions and methods described herein may be used to change T cell profile to alter immunopathogenesis from autoimmune and allergic diseases, cancer, or chronic viral infection or during recovery phase of an acute infection. Autoimmune diseases may be selected from the group consisting of MS, IBD (Crohn's, ulcerative colitis), Celiac's disease, type I diabetes, atopic wheeze, and atopic dermatitis. Types of cancer may be selected from colon, leukemia, pancreatic, prostate, ovarian, breast, or brain cancer.

### DESCRIPTION OF FIGURES

FIG. 1. Systems-level analysis of immune development in human newborns. Monocyte abundance analyzed by Mass cytometry and IFNβ and IL1RA measured by Olink assays in longitudinal blood samples (n=858) from 208 individual children and binned by sampling day of life. Box plots colored by mean rank. CB, Cord Blood. (FIG. 1A) Blood Mass cytometry analyses of memory Tregs, pDC, plasmacytoid DC. (FIG. 1B) blood γδT-cell abundance and subset of γδT-cells expressing CD161 and plasma IL-17A (FIG. 1C). (FIG. 1.D Representative FACS plots of CD38⁺CD62L⁻CD4⁺T-cells sorted at postnatal day 0, 4, 29 and 76 from newborn PBMCs and subjected to bulk mRNA-sequencing. (FIG. 1E) Gene set enrichment analysis showing top enriched hallmark pathways in mucosal-specific vs. total memory CD4⁺ T-cells.

FIG. 2. Bifidobacteriaceae expand after birth. Species level abundances within the Bifidobacteriaceae family. Only species detected in at least one sample shown included.

FIG. 3. Immune system state in infants with low vs. high bifidobacteria. (FIG. 3A) Fold-change immune cell frequencies between 56-152 days after birth in infants with high vs. low gut bifidobactereacea. (FIG. 3B) Fold-change plasma protein levels at 3 months of life in infants with high vs. low fecal bifidobacteria. (FIG. 3C) Spearman correlation matrices of immune cell frequencies in the third month of life in children with high vs. low fecal Bifidobacterium. Black boxes highlight modules of particularly co-regulated immune cell populations, cluster 1. denotes a thymic output cluster of coregulated naïve T-cell subsets and cluster 2. indicates a regulatory cluster involving memory Tregs, activated T-cells and proinflammatory monocytes that differs between children with abundance vs. depleted gut Bifidobacterium.

FIG. 4. B. infantis EVC001 reduces intestinal inflammation in breastfed infants. (FIG. 4A) Fecal cytokines at baseline (Day 6) and post-treatment with B. infantis EVC001, or no supplementation. Cytokines measured as picogram/mg of feces, median values were log-transformed and scaled from 0 to 1. (FIG. 4B) Spearman correlation coefficients between fecal cytokine levels and bacterial abundance. * P=0.05, ** p=0.01, *** p=0.001.

FIG. 5. CD4⁺ T-cell polarization under the influence of microbial metabolites. (FIG. 5A) CD4⁺ T-cell polarization in vitro in the presence of fecal water from infants supplemented with B. infantis EVC001 or control (No supplement). (FIG. 5B) UMAP plots of polarized T-cells analyzed by targeted sc-mRNA-seq. (FIG. 5C) PAGA plots of T-cells polarized in the presence of fecal water from infants given B. infantis EVC001 supplementation or control. Coloring by cell density from grey (low) to red (high). (FIG. 5D) Volcano plot showing differentially expressed mRNA in Th0 cells cultured with decal water from infants given B. infantis EVC001 supplementation or control. (FIG. 5E) Fecal Tryptophan metabolites measured on day 21 from EVC001 treated and control children, p− values indicate mean comparison EVC001 vs. control samples. (FIG. 5F) T-cells polarized as in (B) but instead of fecal water supplemented by ILA (1 mM) or no supplement and mRNA expression in individual cells quantified by targeted sc-mRNA-sequencing. Mean expression from 878/362 cells (Th0), 395/697 cells (Th1), 1073/1922 cells (Th2) and 861/403 cells (Th17).

FIG. 6. Microbiome associations with fecal cytokines. Correlation of day 60 IFNβ concentration and day 21 relative abundance of Bifidobacterium species showed positive correlation with Bifidobacterium longum (R=0.48, P=0.0028). Each cytokine was tested in duplicate. Statistical analysis was completed using Wilxocon rank-sum test. P values were adjusted using Bonferroni-Holm method and considered statistically significant if * P<0.05; ** P<0.01; *** P<0.001; **** P<0.0001.

FIG. 7. Related to FIG. 6. CD4⁺ T-cell polarization under the influence of microbial metabolites and IFNβ. Top genes differentially expressed among Th17-induced states in B. infantis EVC001 treated or control infants fecal water cultures.

FIG. 8 In vitro growth of B. infantis or B. breve on purified pooled HMO, 2′FL or GOS to produce ILA.

### DETAILED DESCRIPTION OF THE INVENTION

The invention disclosed herein describes compositions and methods that provide gut-derived, whether actually derived from the gut or not, signals to 1) promote differentiation of (polarize) naïve T cells to a desired or optimal profile of Th1 subtype and decreased Th2 and Th17 cell subtypes; or 2) to selectively change circulating T cells through induction of a negative regulator to suppress pathogenic T cells such as Th17 cells, as well as Th2 cells. These compositions may be delivered in an ex vivo situation where T cells are removed by apheresis and may be partially or fully purified, treated and returned to body or selected T cell suppression is achieved through an oral, enema or injectable fecal water or a fecal water analog, artificial stool or one of more purified bacterial metabolites. The inventors discovered that naïve T cells and polarized T cell populations can be altered without antibodies or contact with mucosal surfaces. Such compositions that modify T cells may be used to treat a patient in need of altered T cell profiles. A patient may need to change T cell profile to alter immunopathogenesis from autoimmune and allergic diseases, cancer, or chronic viral infection or during recovery phase of an acute infection. In some embodiments, polarization may result from key gut metabolites whether bacteria or host derived or a combination of both delivered ex vivo to a susceptible T cell population, or through intravenous injection. The invention may provide a means of using a mechanism of gut derived oral tolerance without the need for changing the gut microbiome of that patient to achieve a precise outcome. This treatment may be used alone or in combination with products that modulate the gut microbiome directly. Compositions that include probiotics or commensal organisms considered beneficial when fed, the prebiotic or food for the bacteria that may be but not limited to plant glycans (plant oligosaccharides), mammalian milk oligosaccharides, galacto-oligosaccharides or fructo-oliogsaccharides, xylo-oligosaccharides, polydextrose, (PDX) or resistant starch. Such compositions may be fed to deliver a post-biotic benefit. Post-biotic means the result of bacterial activity of prebiotics and probiotics to generate useful metabolites for the host. Examples of postbiotics include i.e. nutrients such as vitamins B and K, amino acids, and substances called antimicrobial peptides that help to slow down the growth of harmful bacteria. Other postbiotic substances called short-chain fatty acids or organic acids help healthy bacteria flourish.

The gut metabolites alone or in combination may stimulate IFNβ mediated pathways resulting in higher detection of endogenous IFNβ in subjects.

An optimal or desirable or protective T cell profile means the polarization of naïve T cells towards a population distribution that is protective against the immune-mediated diseases or infections and includes regulatory T cells and T helper cells in certain ratios with certain cytokines, regulators and cell surface proteins expressed.

Regulatory T cells (Tregs) mean T cells that have a role in regulating or suppressing other cells in the immune system. Tregs modulate the immune system, maintain tolerance to self-antigens, and prevent autoimmune disease. Tregs express the biomarkers CD4, FOXP3, and CD25 and are thought to be derived from the same lineage as naïve CD4 cells.

Helper T cells are types of immune cells that are generally considered essential in B cell antibody class switching, breaking cross-tolerance in dendritic cells, in the activation and growth of cytotoxic T cells, and in maximizing bactericidal activity of phagocytes such as macrophages and neutrophils. Helper T Cells are also called CD4-positive T lymphocytes.

CD4+ T cells function is often characterized by their frequency or ratio. Frequency of proinflammatory T cell subtype (i.e. Th17) may be dysregulated or overabundant and/or frequency of Th2 may be dysregulated or overabundant compared to Tregs or Th1 CD4+ T cells, respectively, and need correction to prevent an autoimmune condition, resolve chronic inflammation or immunopathogenesis from chronic viral infection.

Bacterial metabolites mean one or more outputs of a fermentation process which includes bacteria in a system such as the gut or in a fermentation vat. For example, those produced in commercial processes regardless of size of vessel or those producing a fecal slurry. Bacterial metabolites may be gut derived signals which can include secreted proteins, cytokines, chemokines, peptides, miRNA, primary or secondary bile acids, short chain fatty acids (e.g. butyrate, formate), organic acids (acetate, lactate), tryptophan derivatives, such as indole lactate or serotonin that are used in a polarization composition that may also be called a polarization cocktail or polarization conditioning media.

The polarizing composition of this invention relies on one or more gut-derived signals that polarize T cells without necessarily using specific antibodies to achieve polarization. It may also comprise one or more components that stimulate negative regulators, such as galectin-1 in susceptible T cell populations such as Th2 cells and Th17 cells. The polarizing composition may be used on naïve T cells or in dysregulated T cell populations to alter the T cell profile to a more desirable state.

Gut microbiome means the community of organisms that reside predominantly in the colon but may include organism that reside in the small intestine and may be determined in stool or biopsy samples. The microbiome may be defined by composition and/or functional capacity. Dysbiosis is the lack of certain bacteria, overabundance of potential pathogens or loss of function that may lead to inflammation and disease.

### Collection of Immune Cells or Precursor Cells

Stem cells or naïve T cells can be removed from an individual suffering from, or at risk for an immune mediated disease, such as autoimmune, cancer and allergic diseases. In some embodiments, stem cells or naïve T cells may be harvested from an individual other than the patient (compatible donor T cells).

In some embodiments, stem cells are collected with an apheresis machine from the blood flowing through a catheter, which is inserted into a vein. Blood flows from a vein through the catheter into the apheresis machine, which separates the stem cells from the rest of the blood and then returns the blood to the patient's body. To boost the number of stem cells in the blood, medication that stimulates their production will typically be given for about 4 days beforehand. It can take one to three days to collect enough stem cells for transplant.

In some embodiments, naïve T cells may be collected by apheresis and the white blood cells or leukocytes are collected, and the remainder of the blood is returned to the body. The purification or enrichment of naïve T cells or depletion of other cell types including memory T cells and other non-T cell types may require indirect isolation. In some embodiments, PBMCs are incubated with a cocktail of biotinylated CD45RO, CD14, CD15, CD16, CD19, CD25, CD34, CD36, CD57, CD123, anti-HLA-DR, CD235a (Glycophorin A), and CD244 antibodies. Optionally, biotinylated Anti-TCRγ/δ antibodies can be added for depletion of TCRγ/δ+ T cells. The cells are subsequently magnetically labeled with Anti-Biotin MicroBeads. CD61 MicroBeads are added for concurrent depletion of platelets. [www.jimmunol.org/content/200/1_Supplement/174.38]. In some embodiments, naive T cells are identified by the following characteristics: CD3+CD45RA+CD45RO−CD197+.

In some embodiments, somatic cells are harvested from the patient. Such somatic cells are induced into a pluripotent state. Such pluripotent cells are then transformed into naïve T-cells.

In some embodiments, using methods known in the art, naïve T cells may be produced by inducing pluripotent stem cells [Induction of Pluripotent Stem Cells from Adult Human Fibroblasts by Defined Factors www.cell.com/cell/fulltext/S0092-8674(07)01471-7?_returnURL=https%3A%2F%2Flinkinghub.elsevier.com%2Fretrieve%2Fpii%2FS0092867407014717%3Fshowall%3Dtrue]. In some embodiments, stem cells are collected from bone marrow.

In some embodiments, naive T cells may be harvested for the subject in need or from a compatible donor individual other than the patient in need of T cells preparation.

In some embodiments, naïve T cells or stem cells may be grown in a cell culture.

Embodiments of the herein disclosed invention may utilize T cells or stem cells regardless of the source or method of harvesting. No embodiment describing the source or such T cells or stem cells, or the method of harvesting such T cells or stem cells discussed herein should be read as limiting the scope of the herein disclosed invention.

### Generation of Compositions for Modulating T Cells

The polarizing composition of this invention may be used to change a population of naïve T cells to a desired population of regulatory and helper T cells to make a desired T cell preparation. A T cell preparation of this invention may be defined in terms of the T cell types produced or cell types lost as a result of applying a polarizing composition to said T cells. The polarizing composition applied to stem cells or naïve T cells may be a sterile artificial stool or sterile fecal water. In other embodiments, one or more purified gut metabolites are applied to naïve T cells. In other embodiments, sterile artificial stool, sterile fecal water or purified gut metabolites are applied to a T cell population to polarize naïve T cells or selectively suppress overabundant T cells or reduce the frequency of certain less desirable T cell types. The composition or cocktail making up a fecal water or artificial stool may be used.

An artificial stool of this invention means the supernatant or spent media derived from a fermentation of one or more bacteria, wherein the spent media is separated from the bacterial cells and sterilized to mimic key outputs of fecal water. It may also be considered a post-biotic preparation. The resultant supernatant or spent media may be dried by freeze-drying, spray drying or other means to stabilize the mixture. The supernatant may be concentrated to remove a portion of the water and the liquid used in applications described herein.

Fecal water of this invention, or a water extract of feces means the sterile filtrate removed from the solid material from the feces of a patient with microbiome profile that is known to produce key biochemical and functional attributes. These fecal waters may be further processed to concentrate metabolites, remove certain elements, to further enrich desirable attributes. It may be dried in some instances. In some embodiments, individually sourced metabolites are assembled as a cocktail or composition. These may be assembled as a powder or liquid format.

Gut microbiome means the community of organisms that reside predominantly in the colon but may include organism that reside in the small intestine and may be determined in stool or biopsy samples. The microbiome may be defined by taxonomic composition and/or functional capacity. Dysbiosis is the lack of certain bacteria, overabundance of potential pathogens, or loss of function.

In some embodiments, sterile fecal water is applied to naïve T-cells. In certain cases the sterile fecal water may be applied as an enema or suppository, or in a liquid capsule or other form such as but not limited to a pill, powder, gel, paste, liquid for oral delivery. In other embodiments, a fermentation is used to generate specifically activated cell supernatant. In yet other embodiments, the activated cell supernatant is supplemented with additional host factors to create an artificial fecal or stool water. Sterile fecal water is generated by collecting a stool sample from a non-dysbiotic, healthy breastfed infant donor or suitable donor that has at least 1 g/L, at least 2 g/L, 4 g/L, at least 12 g/L, at least 12 g/L, or at least 15 g/L of one or more oligosaccharides in the diet and has a source of Bifidobacterium. A suitable donor feces may have one or more of the following characteristics: 1) a microbiome that has a relative abundance of at least 20%, at least 30%, at least 40%, at least 50%, at least 60%, at least 70%, at least 80% or at least 90% Bifidobacterium species; 2) relative abundance of at least 60%, at least 70% at least 80% or at least 90% of any B. infantis species; 3) at least 60%, at least 70% at least 80% or at least 90%, an H5 positive B. infantis, such as but not limited to B. infantis EVC001; 4) produces indole lactate (ILA), 5) reduces enteric inflammation; 6) pH less than 5.8, less than 5.5, less than 5.0; 7) overall production of acetate:lactate in ratio approaching 3:2; 8) acetate concentrations of at least 15 μmol/g feces, at least 20 μmol/g feces, at least 25 μmol/g feces, at least 30 μmol/g feces, at least 35 μmol/g feces, at least 40 μmol/g feces, at least 45 μmol/g feces, at least 50 μmol/g feces or at least 55 μmol/g feces or lactate concentrations of at least 2 μmol/g feces, at least 3 μmol/g feces, at least 5 μmol/g feces, at least 10 μmol/g feces, at least 15 μmol/g feces, at least 20 μmol/g feces, at least 25 μmol/g feces, at least 30 μmol/g feces, or at least 35 μmol/g feces or 9) produces IFNβ above a minimum detectable limit to be effective as part of a polarization composition it may be expressed as 0.001 to 100 mmol/kg. Suitability may be determined by the enrichment of HMO utilization genes in the microbiome or expression of key metabolites such as lactate dehydrogenase IV (LDH4). Desirable fecal metabolic profiles may be identified using WO2019/055718 incorporated by reference herein.

Within the genome of B. infantis there are 5 unique gene clusters and over 700 genes unique to this species compared to closely related Bifidobacterium longum species. The H1 cluster includes Blon_2331, Blon_2332, Blon_2334, Blon_2336, Blon_2342, Blon_2343, Blon_2344, Blon_2347, Blon_2348, Blon_2350, Blon_2351, Blon_2352, Blon_2354 and Blon_2355. The H2 cluster (fuscosidase pathways/activities) includes Blon_0243, Blon_0244, Blon_0245, Blon_0246, Blon_0247, Blon_0248 and. The H3 cluster (fucosidase related pathways/activities) includes Blon_0423, Blon_0424, Blon_0425, and Blon_0426. The H4 (sialidase related pathways) includes Blon_0641, Blon_0642, Blon_0643, Blon_0644, Blon_0645, Blon_0646, Blon_0647, Blon_0648, Blon_0649, Blon_0650 and Blon_0651. The H5 (lacto-N-pathways/activities include Blon_2171, Blon_2172, Blon_2173, Blon_2174, Blon_2175, Blon_2176, and Blon_2177. In particular, the presence of Blon_2177 (gene name: extracellular solute-binding protein, family); Blon_2176 (gene name: binding-protein-dependent transport systems inner membrane component); Blon_2175 (gene name: binding-protein-dependent transport systems inner membrane component) can be used to identify bacteria with a functional H5 gene cluster. List of genes that are associated with HMO consumption in B. infantis as described in Locascio 2010 and Garrido (2013) Microbiology 159: 649-664. Functionally equivalent genes from other Bifidobacterium or other genus, families, species of bacteria can contribute to the overall function for certain metabolic pathways deemed important in health or disease and the functions of carbohydrate transport, HMO carbohydrate metabolism. An example of other taxa that carry some of these HMO utilization genes include Bacteroidaceae, Enterobacteriaceae, Enterococcaceae, Eubacteriaceae, Clostridiaceae, Corynebacteriaceae, and Streptcoccacaeae. Akkermansia sp. might also carry HMO related functions.

The composition of Bifidobacterium species in the feces may include one or more of Bifidobacterium may be from species such as B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. pseudocatanulatum, B. pseudolongum. In a more preferred embodiment, B. longum, B. infantis, B. breve, and B. bifidum are found in feces. In a more preferred embodiment, B. infantis is found in feces. The feces may also comprise Lactobacillus species The Lactobacillus may be from species, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius L. paracasei, L. kisonensis., L. paralimentarius, L. perolens, L. apis, L. ghanensis, L. dextrinicus, L. shenzenensis, L. harbinensis. Lactobacillus species have recently been reclassified and one skilled in the art understands the species named herein may have new names but are the same organisms [International Scientific Association for Probiotics and Prebiotics. New names for important probiotic Lactobacillus species. 2020 Apr. 12 [cited 20 Apr. 2020]. In: ISAPP Science Blog [Internet]. Sacramento: ISAPP 2020. Available from: isappscience.org/new-names-for-important-probiotic-lactobacillus-species/.] It is recognized that other organisms that contribute to increased human milk oligosaccharide utilization gene enrichment in a microbiome, whether isolated from suitable donor feces or are engineered to produce effects of such microbiome are included here.

In some embodiments, the feces may contain less than 50% relative abundance, less than 40%, less than 30%, less than 20%, less than 10%, or less than 5% bacteroides. In other embodiments, the feces has a relative abundance of less than 40%, less than 30%, less than 20%, less than 10%, less than 5%, less than 2%, or less than 1% Enterobacteriaceae.

A fecal slurry is generated by adding a suitable liquid, such as phosphate buffered saline (PBS). A fecal slurry may be a 1-50% solution. In a preferred embodiment, the fecal slurry is between a 10-20%, a 20-30%, or 30-40% solution. In more preferred embodiments, the fecal slurry is between 15-20%, 20-25%, 25-30%, or 30-35% solution. The cells and cell debris is removed by a method such as but not limited to centrifugation. The remaining fecal water is sterilized, preferably by a filtration method. The sterile-filtered water may be applied directly with or without dilution to naïve T cells.

A fecal slurry may be used as a starting point for a secondary fermentation to enrich for certain metabolites using MMOs or GOS including any synthetic MMOs described herein (i.e. 2FL, 3FL, 3SL, 6SL, LNB, LNT, LNnT) or one purified from any mammalian source of whey permeate, colostrum or mature milk processing fraction (human, cow, horse, goat, sheep, pig, water buffalo).

In an alternative embodiment, a polarizing composition is produced from fermentation of one or more Bifidobacterium species in specialized culture conditions, such as the secondary fermentation described above. In some embodiments, co-cultures include one or more of selected from Bifidobacterium, Lactobacillus, Bacteroides, Enterobacteriaceae as a basis for an artificial stool. The Bifidobacterium species may be selected from B. infantis, B. longum, B. suis, B. breve, B. adolescentis, B. dentium, B. bifidum, B. pseudolongum and B. pseudocatenulatum. In a preferred embodiment, an activated cell supernatant is produced from fermentation of B. infantis in specialized culture conditions. Exemplary strains include, but are not limited to B, infantis EVC001, B. breve (215W44a), B. adolescentis (ATCC15703), B. dentium (ATCC27534), B. pseudocatenulatum (DSM 20438) B. infantis (KCTC5934, JCM11347 and 257F), B. longum (BBMN68, JDM341).

Mammalian milk oligosaccharide” (MMO) or glycan is defined here as any oligosaccharide that exists naturally in any mammalian milk whether it is its free form or bound to a protein or lipid. MMO and glycans encompass synthetic structures as well as those extracted or purified from sources other than mammalian milk so long as the compound mimics that found in mammalian milk in structure and/or function. That is, while MMOs may be sourced from mammalian milk, they need not be for the purposes of this invention. Sources of MMO may include colostrum products from various animals including, but not limited to cows, goats and other commercial sources of colostrum. It may include MMO enriched from whey permeate, human milk products that are modified through processes such as skimming, protein separation, pasteurization, retort sterilization may also be a source of MMO. MMO includes human milk oligosaccharides.

In some embodiments, the fermentation media includes at least one selective carbon source. In some embodiments, carbon sources may be selected from HMOs including but not limited to LNT, LNnT, 2FL, 3SL, 6SL, bovine milk oligosaccharides (BMO), bovine colostrum (BCO). HMO selected from, but not limited to, natural or synthetically-produced oligosaccharides including lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose (3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. Other glycans include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH) The media may contain tryptophan, whey protein hydrolysate, vitamins, minerals.

Inputs to fermentation drive production of key metabolites that include at least acetate and lactate at a ratio of 3:2 or acetate concentrations of at least 15 μmol/ml feces, at least 20 μmol/ml feces, at least 25 μmol/ml, at least 30 μmol/ml, at least 35 μmol/ml, at least 40 μmol/ml, at least 45 μmol/ml, at least 50 μmol/ml or at least 55 μmol/ml or lactate concentrations of at least 2 μmol/ml, at least 3 μmol/ml, at least 5 μmol/ml, at least 10 μmol/ml, at least 15 μmol/ml, at least 20 μmol/ml, at least 25 μmol/ml, at least 30 μmol/ml, or at least 35 μmol/ml, and the level in the final dried supernatant powder at least a level that meets the desired level in final T cell polarizing composition or T cell conditioning media. The fermentation may be measured for detection of ILA and the level of ILA is determined to be above the threshold for effective polarization of T cells in an ex vivo application.

In some embodiments, expression and/or activity of Endo-beta-N-Acetylglucosaminidase from B. infantis provides function and/or the metabolites required. EndoBI is a glycosylhydrolase that can cleave N-glycans particularly high mannose, hybrid and/or complex glycans. Other endoglycosidases from any source may be used to cleave O-linked glycans. Endo-beta-N-Acetylglucosaminidase from B. infantis (such as EndoBI-1) can cleave N-glycans from glycoproteins. Mannosyl-glycoprotein endo-β-N-acetylglucosaminidases or simply endo-β-N-acetylglucosaminidases (ENGase, EC 3.2.1.96) are glycoside hydrolyses that cleave the N,N′-diacetylchitobiosyl unit in high mannose glycopeptides and glycoproteins containing the -[Man(GlcNAc)₂]Asn- structure. In this invention endo-β-N-acetylglucosaminidases are considered those that are found in B. infantis and recombinant versions of those.

The culture media will contain at least one or more specific HMOs, typically in specific ratios. In some fermentations, one or more of LNT, LNnT, N-acetylglucosamine (NAG), LNB, 2FL, GOS are used as carbon sources.

The output of the bacterial fermentation means the fermentate or supernatant or spent media used as some or all of the polarizing composition and may include one or more of the following indole lactate, acetate lactate. Fermentation by B. infantis produce specific bacterial metabolites, including but not limited to lactate, acetate, indole-3-lactic acid, indole-3-acetic acid, bile acids (cholate, chenodeoxycholate, cholate sulfate), that are beneficial to humans. It may be used in its raw state (no further processing, it may be concentrated, or one or more elements removed by filtration or other means to refine or enrich the spent media). The output of the fermentation may mimic essential elements of a fecal water thus generating an artificial stool. This may be achieved during the fermentation process.

The bacterial fermentate (supernatant or spent media) may be mixed with one or more host factors (gut signals) including but not limited to IFNβ, IL-2, IL-4, IL-5, IL-6, IL-8, IL-10, IL-13, IL-12p70, IL-17A, IL-21, IL-22, IL-27, IL-31, IL-33, MIP-3a, TNFα, TGFβ, IL-1b, and/or IFNγ. In a preferred embodiment, one or more of the following host factors are included IFNβ, IL-2, IL-22, Il-27, TGFβ and/or IFNγ. Bacterial fermentate may be mixed with gut signals at the liquid stage prior to drying, or a dry fermentate may be dry blended with other gut signals. Acetate, lactate, indole lactate and other key metabolites may be sourced individually and mixed at specific ratios to recreate the fecal water without ever having to grow B. infantis. In some embodiments, IFNβ is added to the mixture. The polarizing composition may include this mixture with or without the bacterial fermentate.

In certain instances, the composition may include metabolites or signaling molecules generated as a result of contact with dendritic cell cultures. The output of dendritic cells in culture, including specific combinations of cytokines and chemokines may be used an input to generate artificial stool, to facilitate the production of key gut metabolites for use in fermentation or as part of a polarization composition or conditioning media. Exposure of dendritic cells to potentially pathogenic bacteria or other antigens may be used. In some embodiments, the polarizing composition does not require fermentation step, but comprises a source of ILA (indole lactate), lactate, acetate, TGFβ, IL-2, IL-10, and/or IL-27.

Some embodiments of this invention include the addition of one or more glycans to the gut of subjects in need of immunotherapy. These glycans may come from natural sources or they may be synthetically produced. In some embodiments of this invention the glycan included is a MMO of degree of polymerization (DP) 2-8. In more preferred embodiments of this invention MMO is a HMO. In some embodiments, the glycan is released from a glycoprotein. In some embodiments, the glycan contains at least one residue of fucose or sialic acid. In some embodiments, the glycan contains at least one mannose residue. In other embodiments, the glycan contains at least one N-acetylglucosamine. In some embodiments, the glycan contains galactooligosacharide (GOS) fructoologosaccharide (FOS) or xylooligosacharide (XOS).

In any of the foregoing embodiments, the composition may comprise Bifidobacterium in an amount of 0.1 million-500 billion Colony Forming Units (CFU) per gram of composition. The composition may be in an amount of 0.001-100 billion Colony Forming Units CFU, 0.1 million to 100 million, 1 million to 5 billion, or 5-20 billion CFU per gram of composition. The Bifidobacterium may be in an amount of 0.001, 0.01, 0.1, 1, 5, 15, 20, 25, 30, 35, 40, 45, or 50 billion CFU per gram of composition. The Bifidobacterium may be in an amount of 5-20 billion CFU per gram of composition or 5-20 billion CFU per gram of composition or 0.1 million to 100 million CFU per gram of composition.

Compositions disclosed herein may utilize many different forms and delivery mechanisms including, but not limited to, capsule, packet, sachet, foodstuff, lozenge, tablet, optionally an effervescent tablet, enema, suppository, dry powder, dry powder suspended in an oil, chewable composition, syrup, or gel.

### The Application of Polarization Cocktails to Naïve T Cells

PBMC derived CD3+CD4+CD45RA+ naive T-cells (CD8/14/19/56 negative) into an enriched culture medium including RPMI 1640+10% FBS+NEAA+1% Pen-strep+55 μM β-mercaptoethanol. Cells are added at a concentration of 2×10⁵ cells/ml and pre-washed and resuspended 2.5 ml T-Activator Dynabeads are added to obtain a bead:cell ratio of 1:2.

### Polarizing Compositions

Naïve T cells may be treated with a composition of factors, including but not limited to gut derived metabolites and signaling molecules, cytokines and factors that promote TCR cross-linking until they are programmed to include regulatory T Cells and other helper T cell types in clinical setting.

In some embodiments, the naïve T cell population is differentiated for at least 1 day, at least 2 days, at least 3 days, at least 4 days, at least 5 days, at least 6 days, or at least 7 days to get desired T cell population profile. In a preferred embodiment, the desired T cell population is achieved in 4-6 days. In a more preferred embodiment, the desired T cell population is achieved in 5 days.

In some embodiments, a desired T cell population can be described as decreased expression of Th2 and Th17 CD4+ T-cells. In other embodiments, negative regulators are upregulated in Th0, Th2, and Th17 but not Th1 CD4+ T cells. In some embodiments, the polarizing composition produces a mixture of T regulatory cells and Th1 cells that enable proper development of the immune system in which chronic low, grade inflammation is not quantified. In other embodiments, a higher frequency of Tregs and Th1 cells work to decrease excessive inflammation known as a cytokine storm, limiting damage to the human body. In other embodiments, higher frequency of Tregs and Th1 cells decrease the severity of autoantibodies. In other embodiments, a higher frequency of Tregs and Th1 CD4+ T cells reduces the severity of autoimmune disease progression. In other embodiments, higher frequency of Tregs and Th1 cells reduces immunopathogenesis due to viral, bacterial, or fungal infections.

In some embodiments, the polarizing composition or cocktail will be optimized for suppressing overabundant or reduce the frequency of certain T cells. This may be used on ex vivo T cells, or it may be delivered orally, by enema or by an injection.

In some embodiments, the polarized cells are measured by frequency or abundance in the final population, or the detection of galectin-1.

### The Use of Desired T Cell Population Profiles—Ex Vivo

The recovered polarized T cells, or recovered altered T cell mixture (suppression of overabundant T cells such as Th2 or TH17 cell types), or the polarizing composition are introduced back into the subject to prevent or treat a disease or condition.

In certain embodiments, the polarized T cells may be introduced with IFNβ alone or in combination with other cytokines. IFNβ may be delivered at dose of 0.0001-50 picogram/ml.

In some embodiments, the subject is an infant less than 3 months of age. In some embodiments, the subject less than 3 months of age is a premature infant requiring a rapid change in T cell profile or suppression of overabundant T cells. In some embodiments, the infant may be a preterm infant who may be born with a gestational age of less than 33 weeks, the preterm babies may be an extremely low birth weight (ELBW), very low birth weight (VLBW), or low birth weight (LBW). In some embodiments, the subject is within a week of birth, a newborn within the first 100 days of life, 1-3 months, more than 3-6 months, 6-12 months, 1-3 years, 4-11 years old, 12-15 years, 16-30 years, more than 30 years, more than 40 years, more than 50 years, more than 60 years, more than 70 years, more than 80 years of chronological age.

Compositions and methods described here may be used to prevent, treat, manage symptoms, reduce risk of relapse for autoimmune disease, including but not limited to MS, IBD (Crohn's, ulcerative colitis), Celiac's disease, type I diabetes, atopic wheeze, and atopic dermatitis. Infant conditions may include necrotizing enterocolitis, diaper rash, colic, late onset sepsis,

In the case of allergies including food allergies a protocol may include an elimination diet, recovering naïve T cells, polarizing T cells in the presence of an antigen (or with preexposure of a dendritic cell culture to the antigen in question), recovery of polarized T cells, return to subject and re-challenge with oral antigen.

Chronic viral infection is selected from HIV or Hepatitis A, B, or C or herpes simplex virus (where immunopathogenesis causes illness or disease progression). In some embodiments, utilizing bacterial metabolites and compositions that selectively upregulate negative regulators on Th2 and Th17 cells, and other CD4 T cell subtypes may be used to help a subject inhibit immunopathogenesis progression towards immunodeficiency and prevent serious illness resulting from bacterial or viral infections.

Cancer types include but are not limited to colon, leukemia, melanoma, lung, brain, breast, ovarian, uterine, and pancreatic cancer.

### Example 1—Bifidobacteria-Mediated Immune System Imprinting Early in Life

Using a cohort of 208 individuals in which plasma and fecal samples were collected from birth through the first year an immunological sequence of events, triggered by microbial colonization, that distinguished infants with different gut bacterial compositions was mapped. In a separate cohort, B. infantis EVC001 was fed to healthy infants in a randomized trial and quantified enteric inflammatory biomarkers. The impact of pooled fecal waters from B. infantis EVC001-fed or control infants on naïve CD4+ T cell polarization, and identified a specific bacterial metabolites that had direct impact on the immunoregulation of T cell subsets was tested.

Major Conclusions: HMO utilization gene abundance (Bifidobacterium) correlated with increased regulatory cytokines, IL-27, and decreased systemic inflammation. B. infantis EVC001-fed infants had significantly increased HMO utilization gene abundance versus controls. EVC001-fed infants had significantly increased levels of regulatory cytokines (IFN-b) and decreased enteric inflammation compared to controls. EVC001 metabolites decreased expression of Th2 and Th17 CD4+ T-cells in vitro. B. infantis specific metabolite upregulated negative regulator in Th0, Th2, and Th17 but not Th1 CD4+ T cells.

Specifically, memory CD4+ T-cells expressing CD38 and lacking the lymphoid tissue homing marker CD62L are mucosal-specific T-cells in humans and found that the most enriched hallmark pathways in the mucosal-specific memory CD4+ T-cells were type-I and type-II IFN responses. Together these results indicate that mucosal-specific memory CD4+ T-cells see antigen after birth (as indicated by the downregulated CD45RA expression), expand in the blood in the newborn intestinal immune system. Shown here correlated infant microbiome function with systemic inflammatory profile. Notably, as HMO utilization gene abundance increased, that is where there was increased CPM values for specific Bifidobacterium gene clusters that are required to capture and metabolize HMOs, there were stark decreases in systemic inflammatory markers including, IL-6, TNFα, IL-17A, and IL-13. Conversely, IL-27, a cytokine with regulatory function was significantly increased. Taken together, these correlative data are suggestive of a link between functional capacity in the microbiome of infants that are reflective in the host's systemic inflammatory profile. Important differences in relative abundance of HMO utilization genes in a randomized, controlled study in which age-matched infants were discordant on B. infantis EVC001 colonization compared to controls. High relative abundance of genes associated with HMO utilization was identified in the microbiomes of infants given EVC001. At day 60 postnatal, the inventors identified significant decrease in enteric cytokines, IL-13, IL17a, IL-21, IL-23, IL-31, IL-33, and MIP-3a in infants colonized with EVC001 compared to those that were not. Conversely, EVC001-fed infants produced significantly more IFNβ, a regulatory cytokine, compared to controls. Additionally, correlation between major taxa in all infants showed a significant correlation between Enterobacteriaceae and Clostridiacea with increased proinflammatory cytokines, while Bifidobacteriaceae abundance was negatively correlated with inflammation. Importantly, Bifidobacteriaiceae abundance was the only taxa that correlated with increased production of IFNβ. Using fecal waters from infants colonized with B. infantis EVC001 and those that were not, the inventors evaluated the direct effect on polarization of naïve T cells. Here the inventors show that EVC001 fecal water skews polarization towards a Th1 phenotypes, while control fecal waters skew polarization towards Th17 and Th2. Furthermore, a bacterial metabolite that is made in high concentration in infants that are colonized with B. infantis EVC001 significantly upregulated the negative regulator, Galectin 1, in naïve, Th2, and Th17 T cells but did not signal the same expression in Th1 cells.

This work combines longitudinal systems immunology analyses and metagenomic profiling of 208 infants born in Stockholm, Sweden and find that depletion of bifidobacteria, and HMO-utilization genes from the fecal metagenome is associated with markers of both systemic and intestinal inflammation and immune dysregulation during the first months of life. The inventors also demonstrate a silencing of intestinal inflammation in breastfed infants in California fed B. infantis EVC001, a strain harboring all fully functional HMO-utilization genes. Fecal water from EVC001 supplemented infants skewed T-cell polarization in vitro away from a Th2 state, and towards a Th1 state and indole-3-lactic acid, 10-fold more abundant in feces from EVC001 treated children vs. untreated controls, upregulated immunoregulatory Galectin-1 in Th2 and Th17 cells during polarization in vitro. This molecular mechanism provides a functional link between beneficial microbes, their metabolites and immunoregulation during the first critical months of life.

### Results

Sequential waves of immune cell expansion during the first months of life. Longitudinal blood samples were analyzed (n=858) from (n=208) infants born at the Karolinska University Hospital between April 2014 and December 2019 (Olin et al., 2018; Pou et al., 2019). Mass cytometry and a panel of 44 antibodies was used (Supplementary Table 1) targeting activation and differentiation markers across 64 blood immune cell populations and quantified 355 unique plasma proteins using Olink assays (Olink, Uppsala, Sweden) (Lundberg et al., 2011) and together these data elucidated developmental immune system changes postnatally.

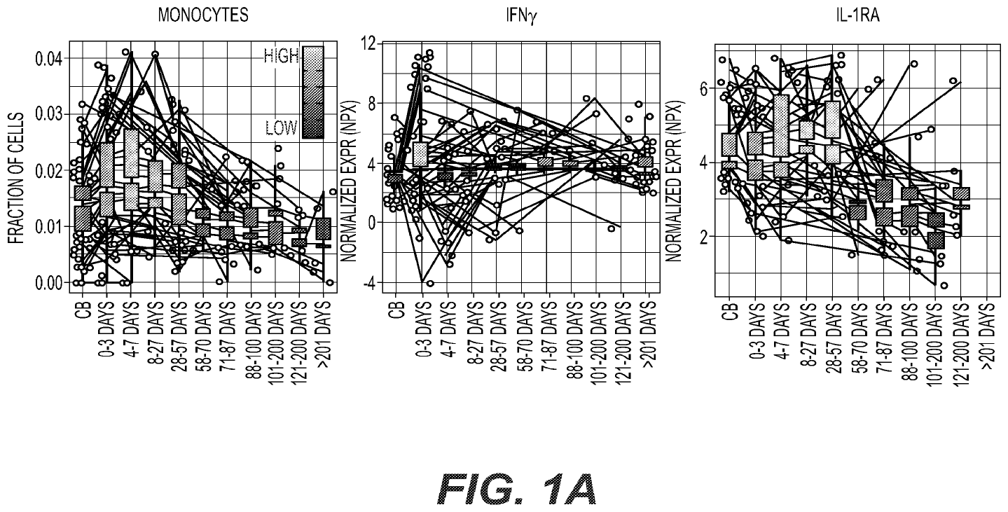

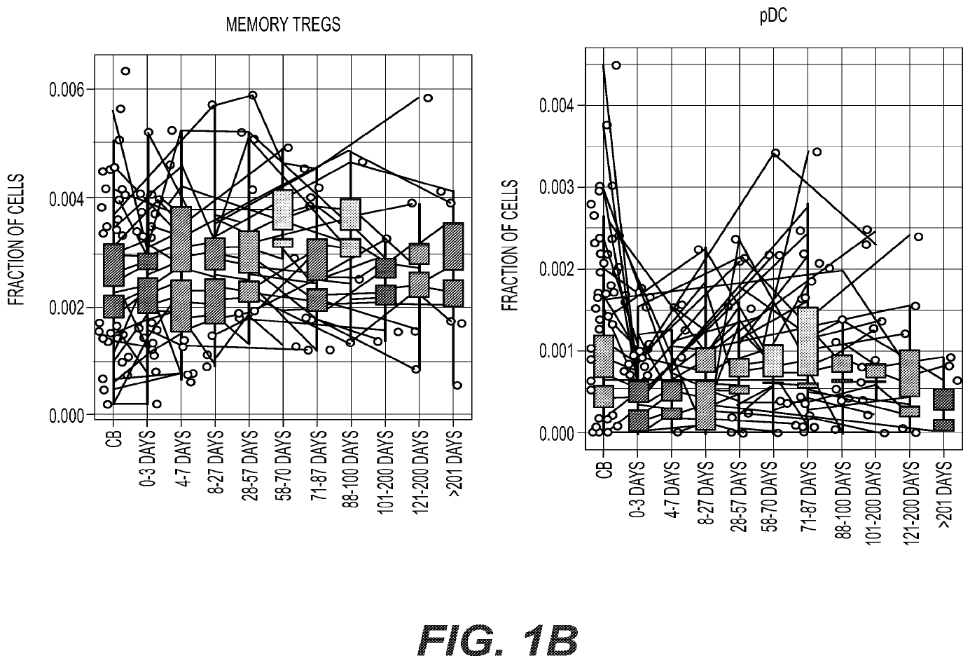

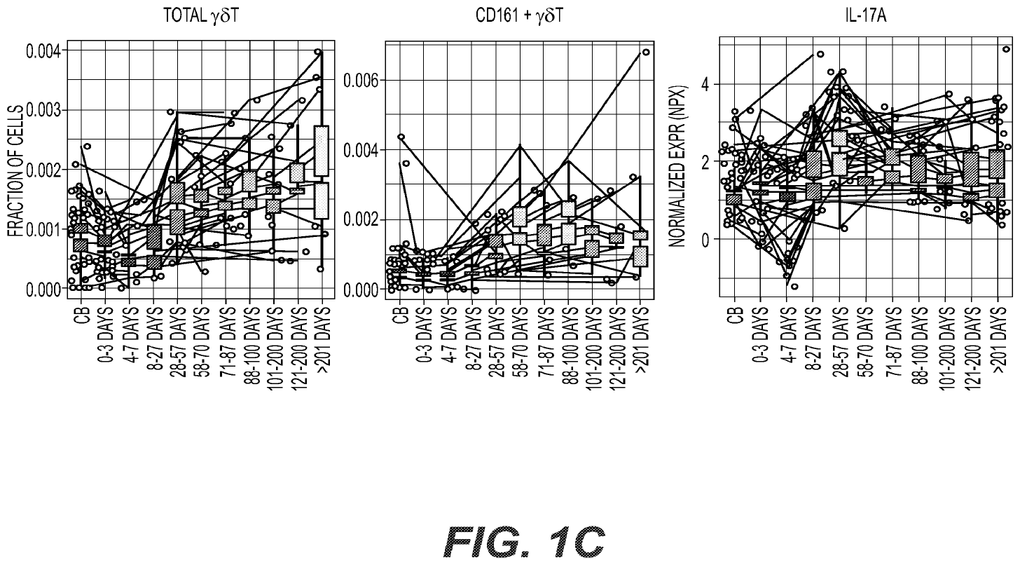

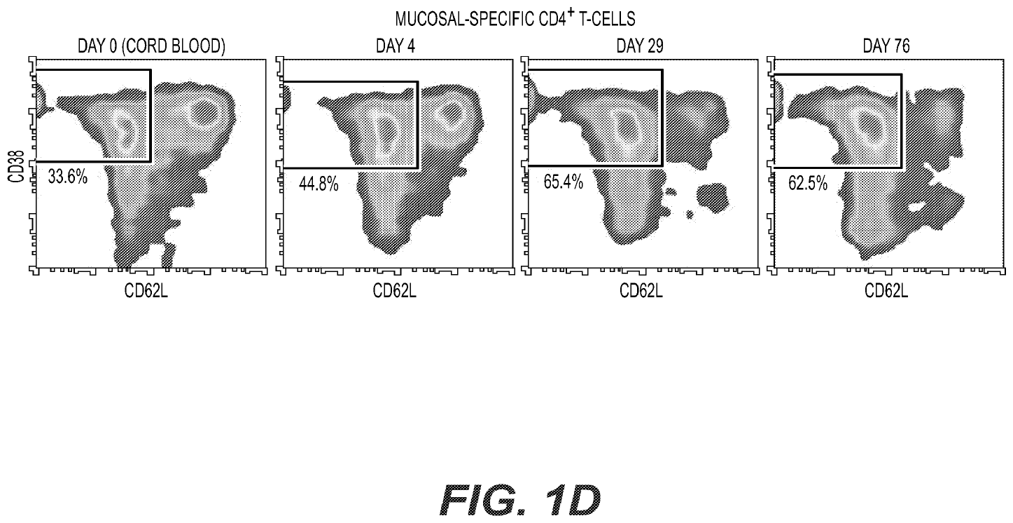

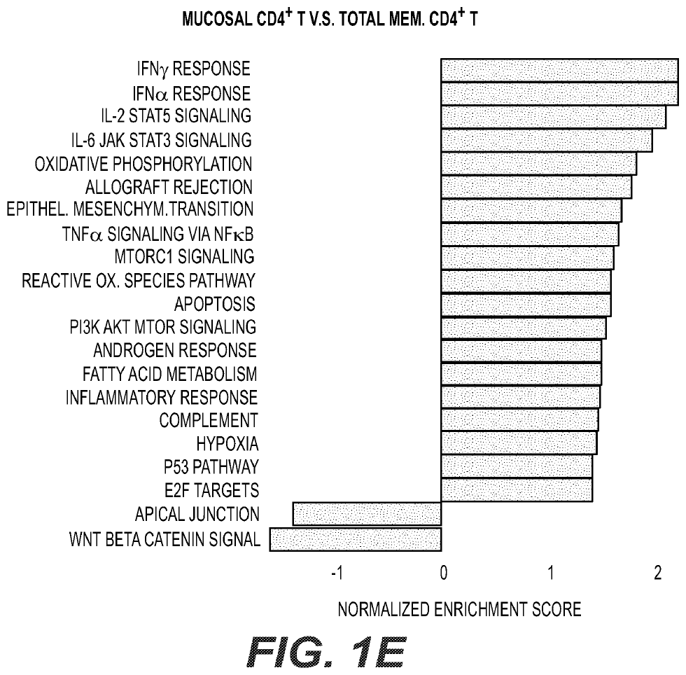

When ordering immune cell frequency measurements by day of sampling, the inventors observed an initial innate response illustrated by the expansion of circulating monocytes that peaked 4-7 days after birth and a transient surge in circulating IFNβ day 0-3 as well as elevated levels of circulating IL1RA, a natural inhibitor of the pro-inflammatory IL-1b, likely having a dampening effect on the initial innate response to microbial exposures during the first weeks after birth. Following the initial monocyte expansion, the inventors observed a gradual increase in the frequency of memory Tregs frequency during the first weeks of life (FIG. 1A). The inventors also found a previously unrecognized contraction and subsequent expansion of plasmacytoid Dendritic cells (pDC) after birth (FIG. 1B. From one month onwards there was a robust increase in circulating γδT cells, especially within the CD161⁺ subset of γδT cells (FIG. 1C). These cells are important producers of IL-17A (Maggi et al., 2010), and increased plasma levels of IL-17A was seen during the same first 2-month time window albeit transiently (FIG. 1C). These findings indicate transient innate and adaptive immune responses but also highlight key regulatory mechanisms at play in early and mid-infancy. These responses are presumably triggered by colonizing microbes similar to the weaning reaction described in mice (Nabhani et al., 2019), but not triggered by weaning and noticeably different in postnatal timing as well as immune cells and proteins involved.

### Expansion of Mucosal-Specific CD4⁺ T-Cells in the Blood of Newborn Children.

Previous work has revealed that immune cells primed by antigens at mucosal surfaces circulate and are detectable in peripheral blood. Specifically, memory CD4⁺ T-cells expressing CD38 and lacking the lymphoid tissue homing marker CD62L are mucosal-specific T-cells in humans, originate in the intestine and are identifiable in blood at a frequency of ˜4-8% of total CD4⁺ T-cells (Pre et al., 2011). Here, the inventors identified this subset of memory CD4⁺ T-cells in the blood of newborn children; but these were more abundant and expanded during the first weeks of life to dominate the circulating memory CD4⁺ T-cell pool (FIG. 1D). These cells have downregulated CD45RA and likely seen antigen, presumably at mucosal surfaces in the intestine, and undergone memory phenotype transition.

To understand these mucosal-specific memory T-cells better, flow cytometry sorting and bulk mRNA-sequencing was used. Total memory CD4⁺ T-cells from the same samples and calculated enriched blood-transcriptional modules (BTMs) (Li et al., 2013)(FIG. 1E). The most enriched hallmark pathways in the mucosal-specific memory CD4⁺ T-cells were type-I and type-II IFN responses (FIG. 1E), and the most upregulated individual genes included Complement regulatory Factor H (CFH), the cytokine IL-15, important for NK cell homeostasis, as well as the monocyte growth factor CSF1 (M-CSF). Together these results indicate that mucosal-specific memory CD4⁺ T-cells see antigen after birth, expand in the blood and interact with NK-cells and monocytes in the newborn intestinal immune system.

Variable Microbiome Colonization of the Infant Gut after Birth.

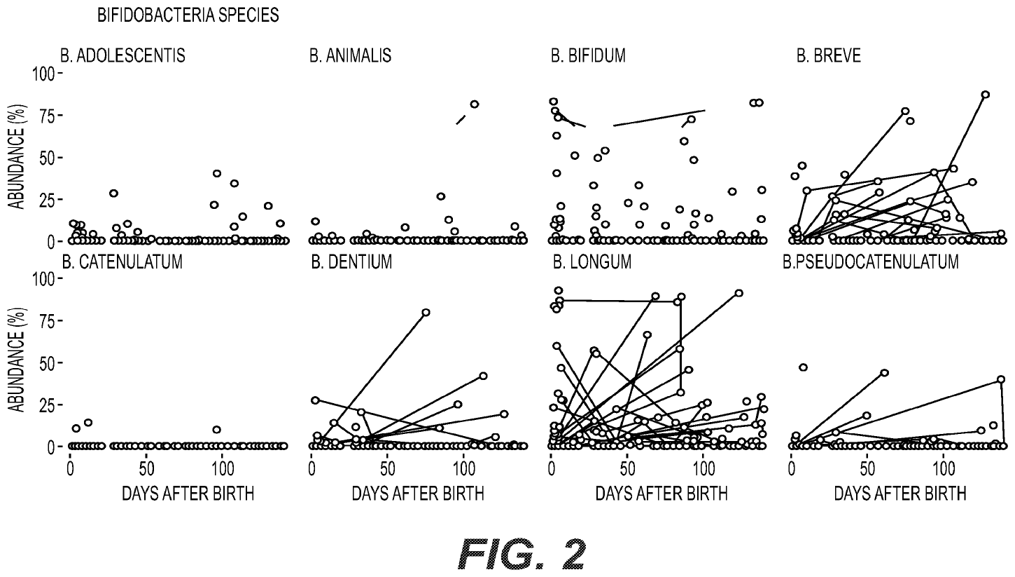

To better understand the microbial antigens driving immune responses after birth the inventors performed shotgun metagenomic sequencing of longitudinal fecal samples (n=347) from 157 of the 208 infants in this cohort. Gut bacterial composition was highly variable at birth but increasingly converged over time. At the family level, there was an increase in Bacteroidaceae and Bifidobacteriacea after birth in the majority of newborns. Bifidobacteriaceae expanded primarily in breastfed infants without antibiotic exposure. This expansion was observed to involve multiple species of Bifidobacteriaceae, but most frequently B. longum, B. breve, and B. bifidum (FIG. 2).

Immune System States Associated with Expanded Gut Bifidobacteria Early in Life.

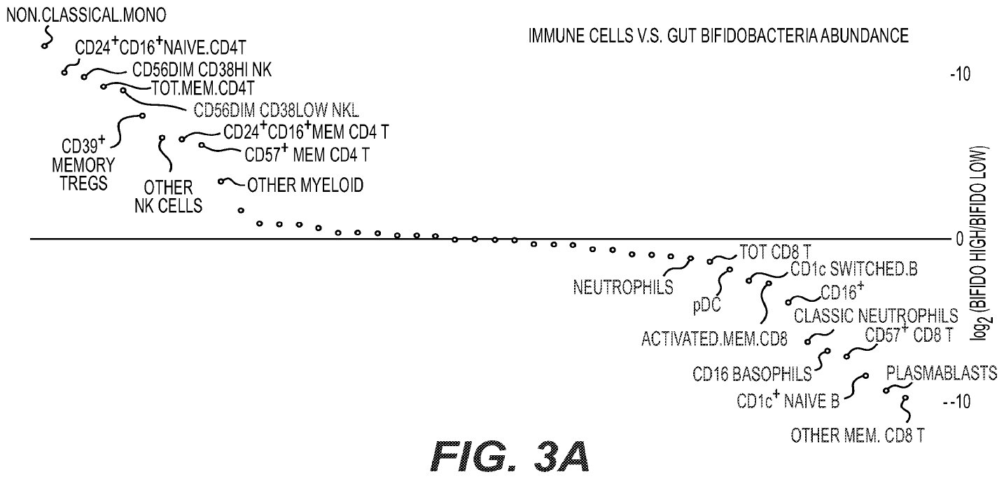

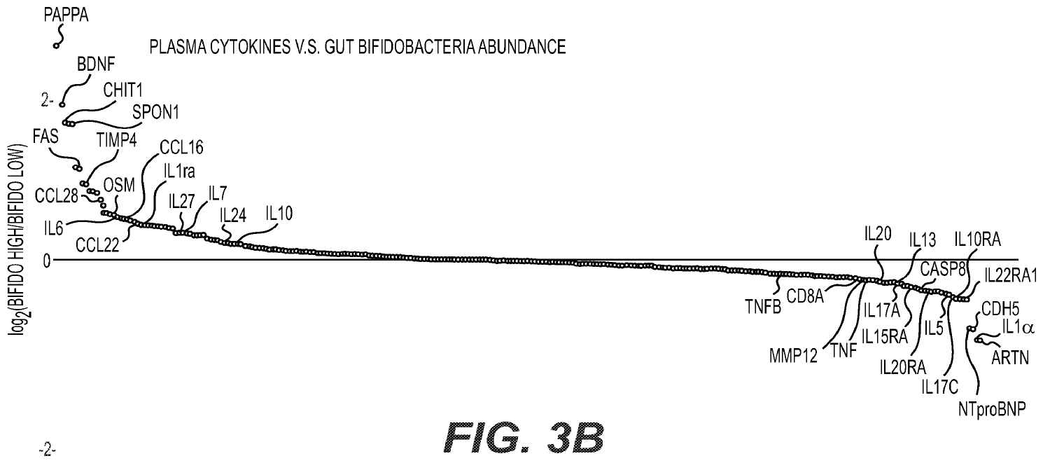

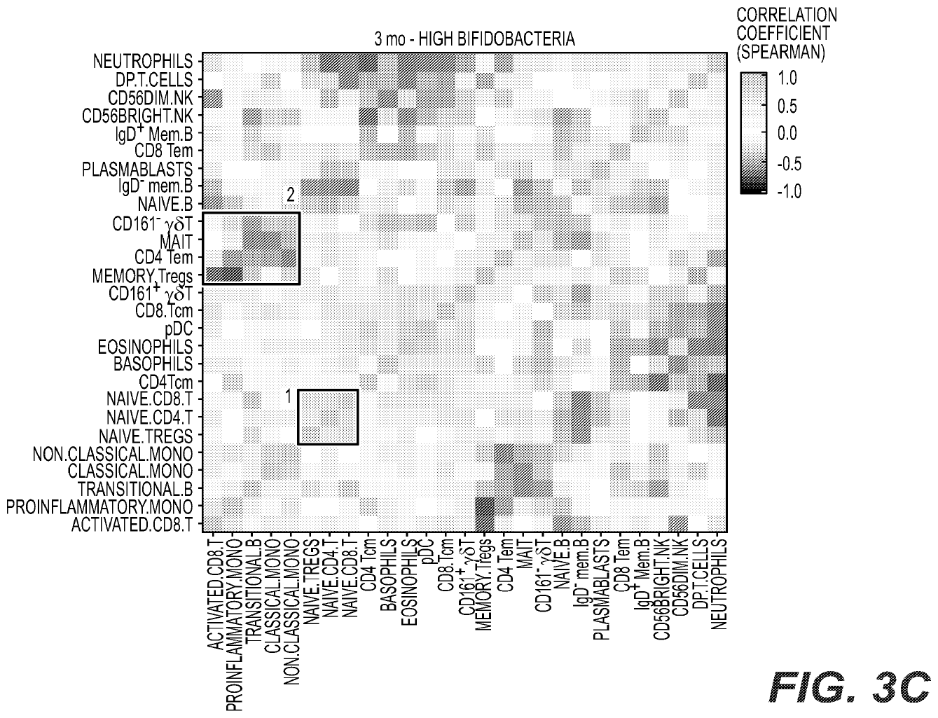

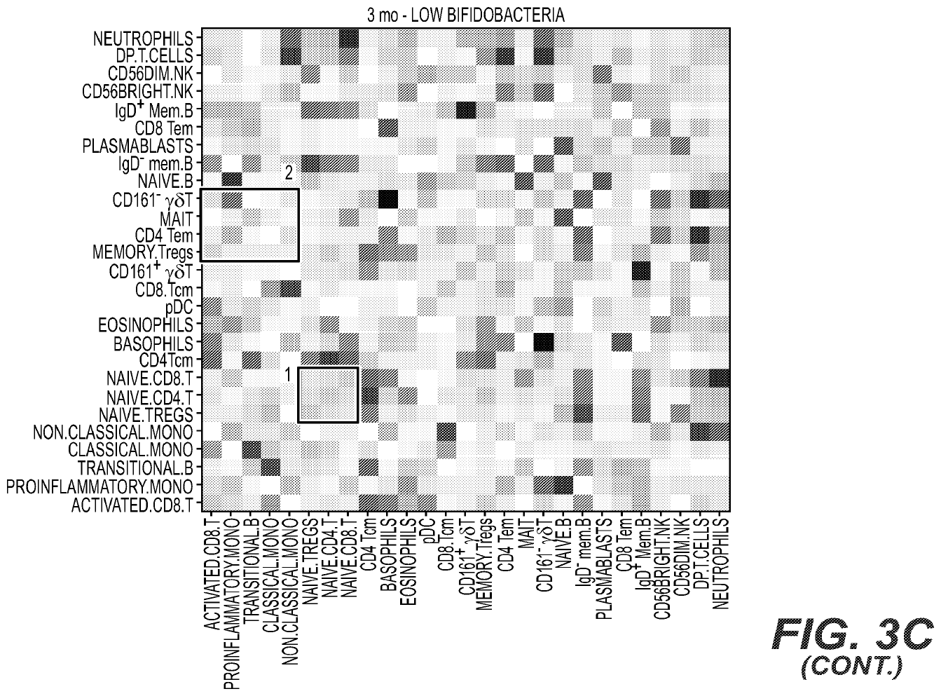

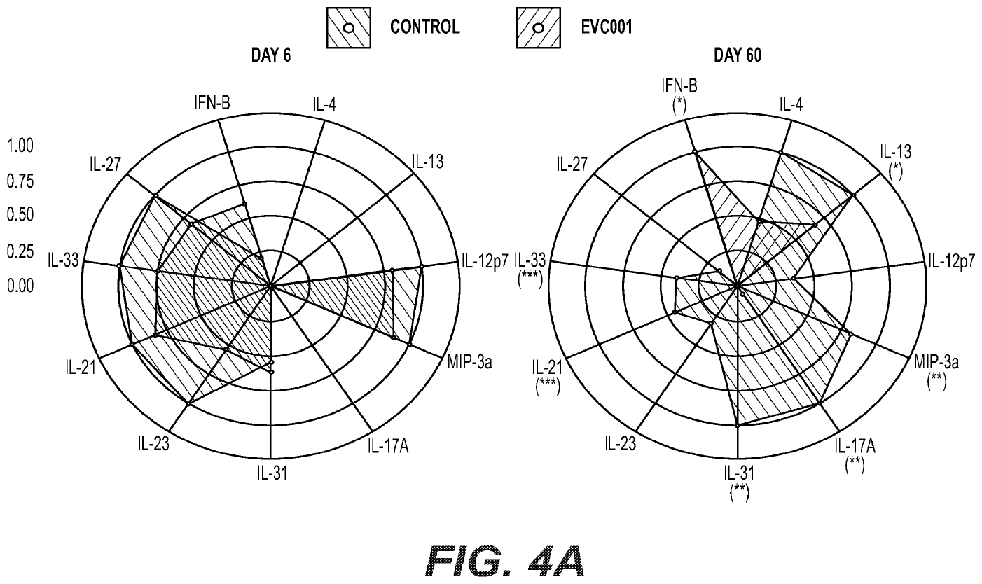

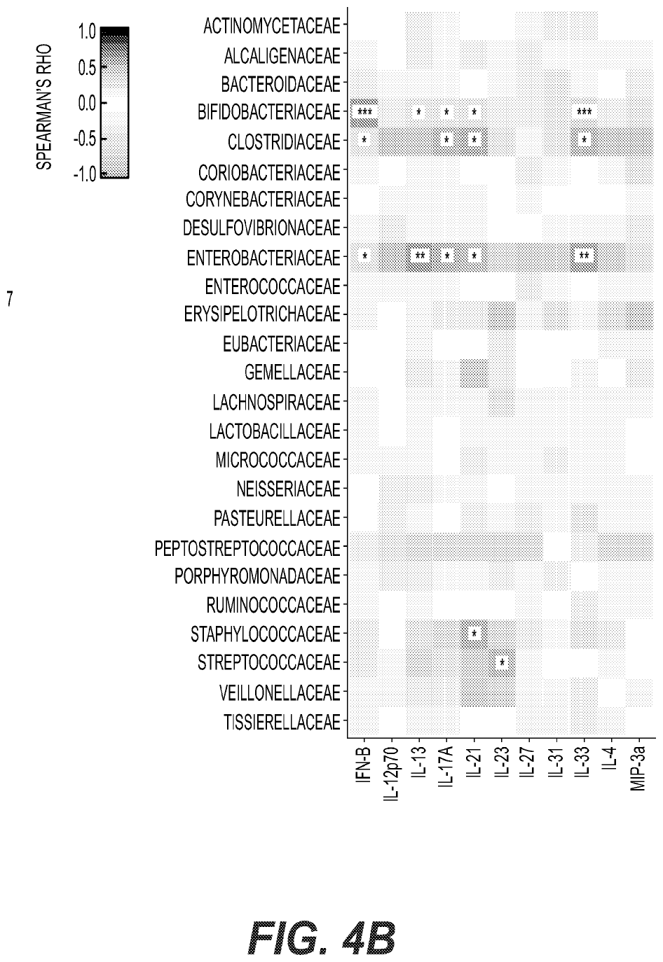

Given the variable expansion of Bifidobacteriacae among newborn children, the inventors compared immune system states between infants with abundant Bifidobacteriacae and those failing to expand such bacteria. Lacking Bifidobacteriacae was associated with expanded populations of neutrophils, basophils, plasmablasts and memory CD8⁺ T-cells indicating both innate and adaptive immune activation (FIG. 3A). MAIT-cells are important T-cells in the intestinal response to bacterial Vitamin B-metabolites (Ioannidis et al., 2020) and these were also more abundant in blood from children with low abundance of Bifidobacteriacea (FIG. 3A).

Conversely, children with abundant gut Bifidobacteriacea had abundant non-classical monocytes, often considered as anti-inflammatory (Narasimhan et al., 2019), as well as antigen-experienced regulatory T-cells expressing the CD39 receptor, a highly suppressive Treg subset (Gu et al., 2017)(FIG. 3A). Also, plasma proteins differed between these groups and children lacking Bifidobacteriacea had elevated levels of TNFα and IL-17A critical mediators of intestinal inflammation, and also the Th2-cytokines IL-13 and IL-1β, serving as an alarmin released by necrotic cells and synergizing with TNFα in a variety of inflammatory responses (FIG. 3B). Children with abundant Bifidobacteriacae had elevated levels of Treg-associated cytokines IL-27 and IL-10 as well as the endogenous IL-1 inhibitor, IL1RA presumably regulating innate IL-1β-mediated responses (FIG. 3B). More surprising was the elevated IL-6 in children with abundant Bifidobacteriacea (FIG. 3B).

To better understand the regulatory cell-cell relationships in the newborn immune system, Spearman correlation matrices was calculated and compared infants with high levels of high and low abundance of Bifidobacteriacae (FIG. 3C). Such cell-cell correlations indicate co-regulated cell populations and allow for context-dependent perturbations to such relationships to be uncovered (Rodriguez et al., 2020). The inventors found strong positive correlations among naïve CD4⁺T-, naïve CD8⁺ T- and naïve regulatory T-cells (cluster 1), likely reflecting overall thymic output and these relationships were comparable among children irrespective of gut Bifidobacteriacea abundance (FIG. 3C). Other modules of co-regulated cell populations were strikingly different between these groups of children. In particular, memory Tregs was inversely correlated with activated CD8⁺ T-cells, proinflammatory monocytes (cluster 2) in children with abundant Bifidobacteriaceae, but this relationship was disrupted in infants lacking such microbes (FIG. 3C). The inventors concluded that infants not colonized by Bifidobacteriaceae or in cases where these microbes fail to expand during the first months of life there is evidence of systemic and intestinal inflammation, increased frequencies of activated immune cells, reduced levels of regulatory cells indicative of systemic immune dysregulation.

### Human Milk Oligosaccharide Metabolism Influences Immune System Development.

Various organic molecules produced by bacteria, including organic acids, phenylalanine and tryptophan derivatives have been shown to broadly influence host health (Ehrlich et al., 2020; Fukuda et al., 2011; Laursen et al., 2020). Specifically, Bifidobacterium-derived metabolites have been shown to modulate pathogen-induced inflammation via the aryl hydrocarbon receptor (AhR) and NRF-2 pathway (Ehrlich et al., 2020; Meng et al., 2020). To test whether the presence of HMO utilization genes could explain the immune perturbations in children lacking gut Bifidobacteriacea, the inventors used the KEGG Orthology (KO) database and identified 57 representative key functions necessary to metabolize HMOs (Nguyen et al., 2021; Sela et al., 2008). Next, the inventors assessed the presence of these HMO utilization genes in fecal metagenomes collected from the infants in our study. The inventors found that the H5 cluster of HMO utilization genes were most commonly detected, but the abundances (counts per million, CPM) of HMO utilization genes were generally low. The inventors then compared relative amounts of the 57 HMO utilization genes to the 355 plasma proteins measured in blood. Here, the inventors observed significant correlations (Spearman) between a number of plasma proteins for example, IL-6, TNFα, IL-17A, and IL-13 levels which were all negatively associated with the presence of HMO utilization genes, in particular genes within the H5 cluster (IL-13, P=7.79e⁻⁸; IL-6, P=1.56e⁻⁶ and TNF, P=8.85e⁻¹⁷). Conversely, infants effectively metabolizing HMOs had elevated levels of IL-27 (P=1.36e⁻¹⁷) a cytokine known to limit Th2 and Th17-type responses in favor of Th1 and regulatory T-cell function (Yoshida and Hunter, 2015).

B. infantis EVC001 Feeding Silenced Intestinal Inflammation Early in Life

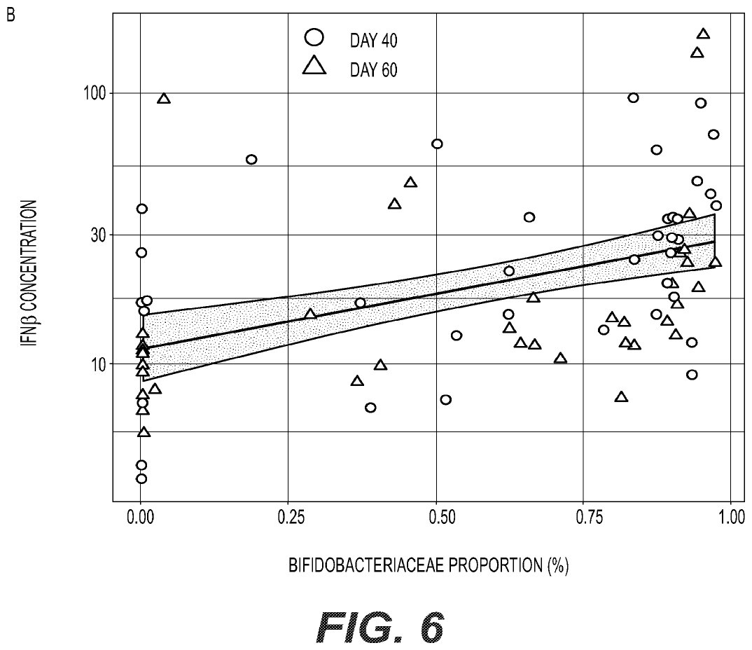

The data above indicate that HMO utilization genes expressed by Bifidobacteria and other beneficial microbes in breastfed infants correlate with decreased systemic inflammation and a reduction in Th2 and Th17-type responses. Importantly, no isolates from any of the infants in the Swedish cohort expressed all HMO utilization genes. To assess the beneficial effects of HMO utilization gene expressing microbes, the inventors used an optimized strain of Bifidobacterium possessing all HMO utilization genes. The inventors supplemented this B. longum supsp. infantis EVC001 to 40 breastfed infants in a second cohort in California. Half of the infants were fed 1.8×10¹⁰ CFU daily from day 7 to day 28 and half were given no supplementation. Fecal samples were collected at baseline (day 6) and on day 60. As expected, all HMO utilization genes were abundantly expressed in the metagenomes of EVC001 treated, but not control children. Analyses of the microbiome composition showed no significant difference at baseline between these groups; however, by day 60 there was a significant decrease in alpha diversity (P=0.0001; Wilcoxon), in B. infantis EVC001-fed infants as compared to controls (FIG. 6).

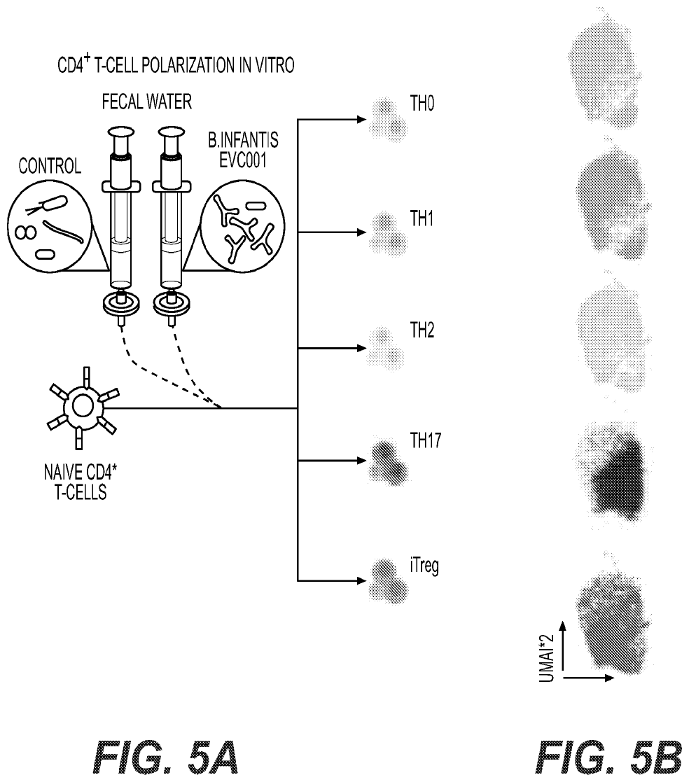

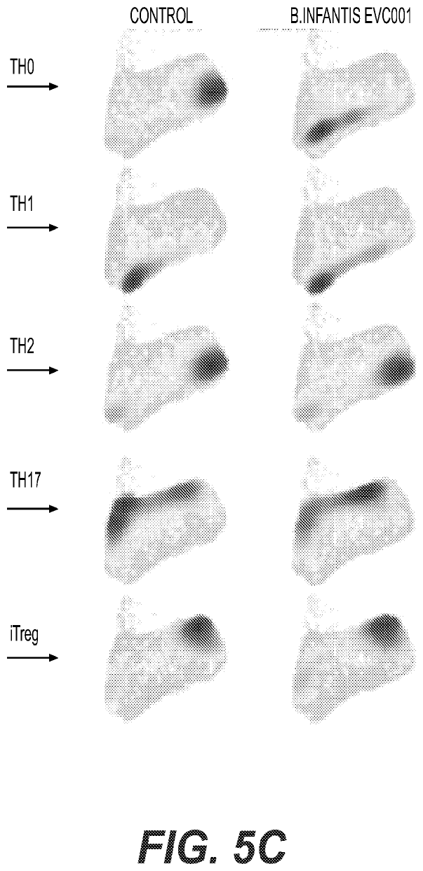

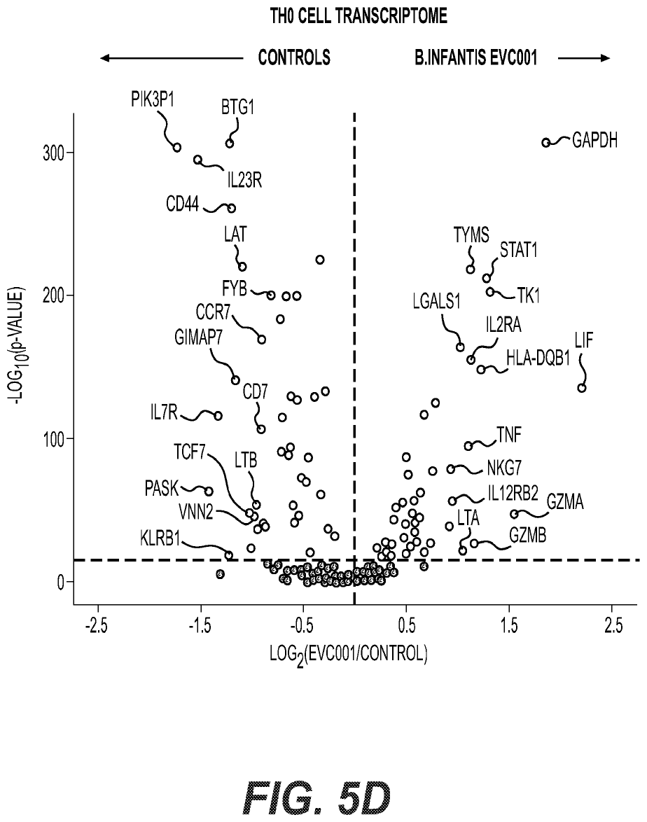

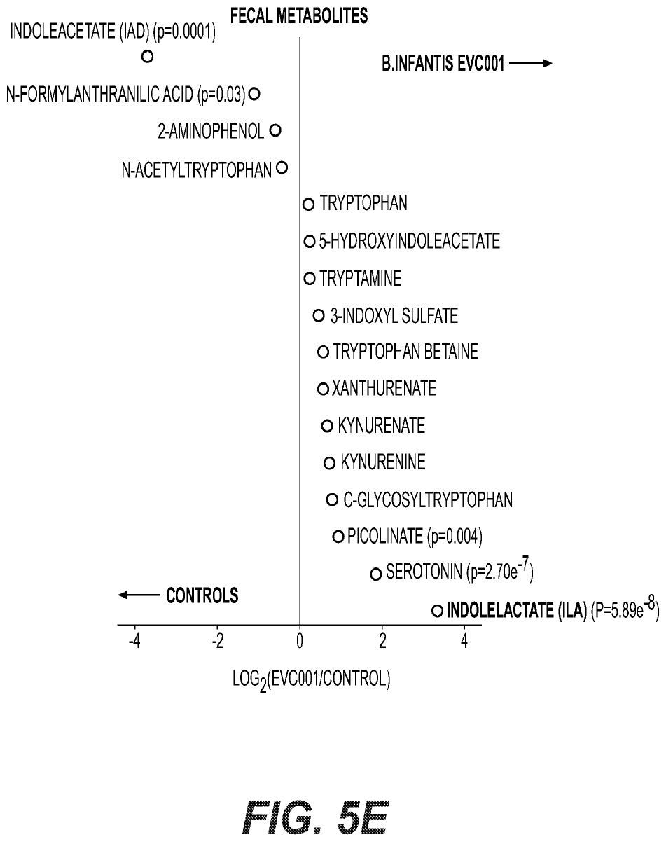

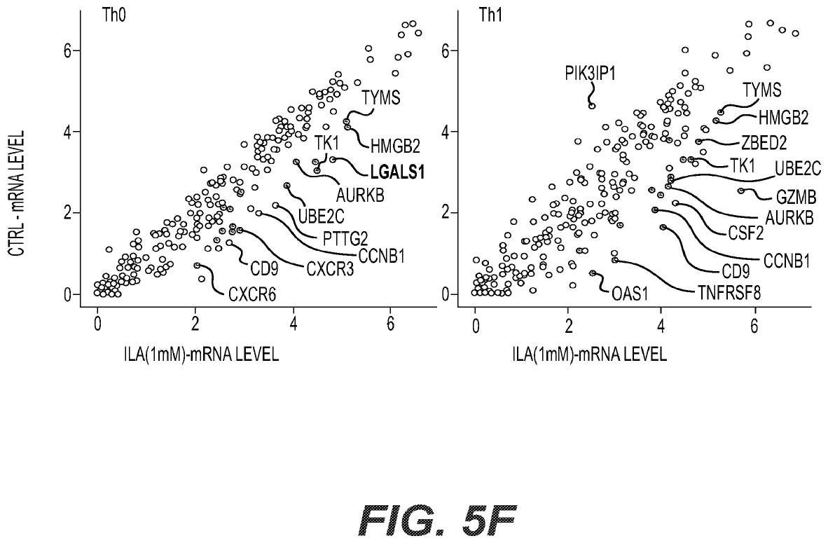

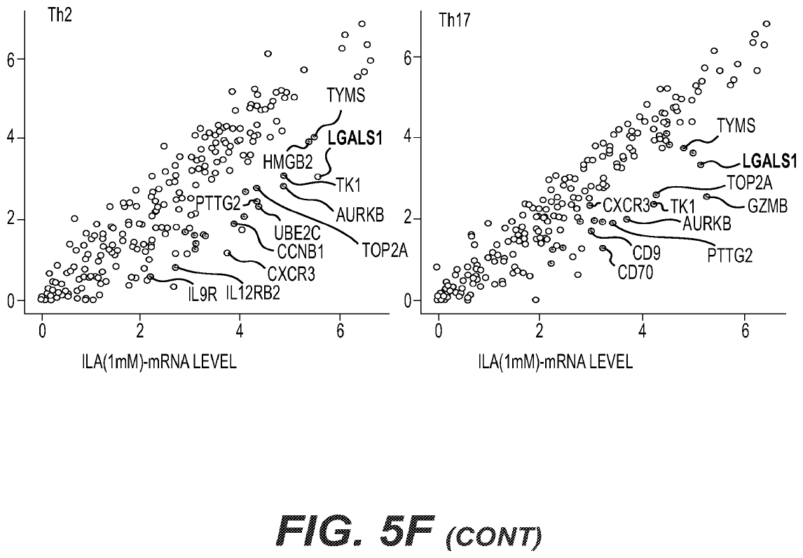

The inventors then measured fecal cytokine levels in these children, and found no significant differences at baseline, but at day 60, infants fed B. infantis EVC001 had reduced levels of IL-13, IL-17A, and regulatory and chemotactic cytokines, IL-21, IL-31, IL-33, and MIP3a (FIG. 5C; Table 2, P=0.015, 0.0029, 0.00066, 0.007, 0.00011, and 0.0078, respectively). Conversely, there was a significant increase in fecal IFNβ in infants fed B. infantis EVC001 (FIG. 5C; P=0.016) and this IFNβ concentration correlated with the abundance of Bifidobacteriaceae (FIG. 6). The inventors performed pairwise correlation tests between individual stool taxa (n=40, day 60) and fecal cytokine concentrations (Spearman, Benjamini-Hochberg false discovery rate, α=0.05) and identified three taxa; Clostridiaceae, Enterobacteriaceae, and Staphylococceae significantly associated with proinflammatory cytokine production. Specifically, Clostridiaceae correlated with IL-17A, IL-21, and IL-33 P=0.018, 0.016, and 0.028, respectively) and decreased IFNβ levels P=0.028), Enterobacteriaceae correlated with elevated IL-13, IL-17A, and IL-33 production (P=0.002, 0.017, 0.036, and 0.005, respectively) and decreased IFNβ levels (FIG. 5D, P=0.049), while Staphylococceae correlated with higher levels of IL-21 (P=0.028) and Streptococcaceae correlated with higher levels of IL-23 P=0.044). In contrast, only Bifidobacteriaceae was associated with elevated IFNβ (P=0.001) and negatively correlated with levels of IL-13, IL-17A, IL-21, and IL-33 (P=0.041, 0.017, 0.027, and 0.001, respectively). The inventors concluded that beneficial microbes expressing all HMO utilization genes in breastfed infants silence intestinal Th2 and Th17 responses. the inventors also describe a previously unrecognized induction of IFNβ by beneficial microbes, although the direct effect on T-cells remain elusive. Thus, it is not the simple presence of bifidobacteria that is responsible for the immune effects, but the metabolic partnership between the bacteria and HMOs.

B. infantis EVC001 Fecal Water Skew T-Cell Polarization

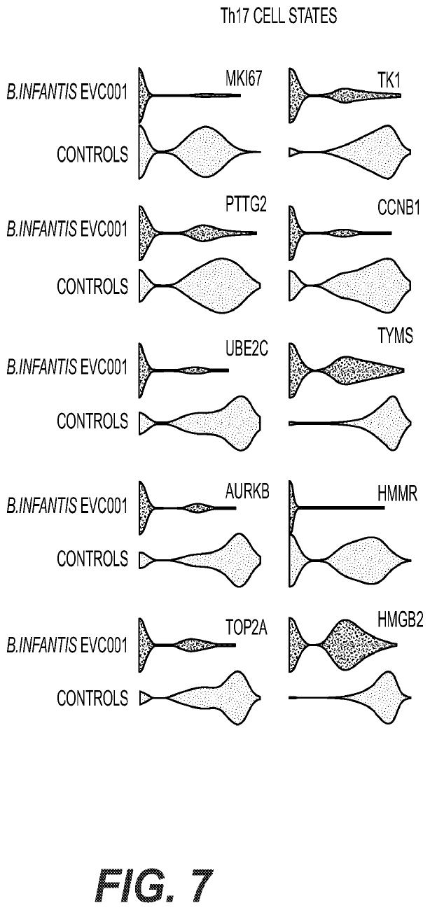

To better understand the direct effects of bifidobacterial metabolites and enteric cytokines on T-cells, the inventors flow-sorted naïve CD4⁺ T-cells from a healthy adult donor and polarized these cells using standard cytokine combinations (Cano-Gamez et al., 2020). The inventors also added fecal waters (1:100 dilution) collected from either B. infantis EVC001 supplemented or control children lacking B. infantis (FIG. 5A). To evaluate the cellular states of polarized T-cells, the inventors applied a targeted multiomics approach quantifying 259 mRNA molecules with known functions in T-cells and 10 surface proteins detected by oligo-coupled antibodies (Abseq™, Rhapsody™, BD Biosciences) (Mair et al., 2020). The UMAP embeddings of cells were largely similar across Th0, Th1 and Th2 conditions but slightly different for Th17 and iTreg states (FIG. 5B). Using agraphical abstraction and clustering method, PAGA (Wolf et al., 2019), we compared the T-cell states by polarizing condition, and, importantly, uncovered differences between T-cells polarized in the presence of B. infantis EVC001 or control fecal water (FIG. 5C). Induced Th1, Th2 and iTreg polarized states were comparable between B. infantis EVC001 and control fecal water cultures (FIG. 5C). Th0 cells on the other hand, cultured without any polarizing cytokines, in the presence of fecal waters from control infants lacking B. infantis, assumed a Th2-like state, while fecal water from infants fed B. infantis EVC001 induced a Th1-like state in these Th0 (FIG. 5C). Differentially expressed genes involved Th1-associated GZMA, GZMB, TNF and STAT1 in cells exposed to B. infantis EVC001 fecal water, while IL23R was highly overexpressed in cells exposed to control fecal water (FIG. 5D). The inventors supplemented Th0 cultures with IFNβ only, but this did not replicate the effect on Th1/Th2 skewing (FIG. 7).

Apart from this skewing towards Th1, the inventors also noted a difference in Th17-polarized states in B. infantis EVC001 fecal water cultures (FIG. 5C). Specifically, naïve T-cells polarized towards Th17 in the presence of fecal waters from control infants had elevated markers of activation and proliferation. Markers such as Ki67 when compared to cells polarized towards Th17 in the presence of B. infantis EVC001 fecal water (FIG. 7).

Collectively, these findings suggest that B. infantis EVC001 metabolites or enteric cytokines induced by the presence of B. infantis EVC001 exert a polarizing effect on naïve CD4⁺ T-cells that favor Th1 polarization, corroborating a mechanism of a silencing effect on fecal IL-13 and IL-17 in vivo.

### Example 2 B. infantis EVC001 Metabolite Indole-3-Lactic Acid Induce Galectin-1 on Th2 and Th17 cells

Next, the inventors assessed fecal metabolites in samples collected from infants fed B. infantis EVC001 and controls infants respectively. A total of 564 biochemicals were significantly different between these fecal samples. Metabolites within the tryptophan metabolism pathway were particularly enriched. The most overrepresented tryptophan metabolite in EVC001-fed infant feces compared to controls was Indole-3-lactic acid (ILA) (FIG. 5E; P=5.89×10⁻⁸, FDR) (Ehrlich et al., 2020; Meng et al., 2020). Importantly, bifidobacteria-derived ILA has recently been shown to bind both the Aryl hydrocarbon Receptor (AhR) and the hydrocarboxylic acid receptor 3 (HCAR3) and modulate monocyte responses to Lipopolysaccharide (Laursen et al., 2020). CD4⁺ T-cells do not express the HCAR3 receptor, but do express the AhR (Uhlen et al., 2019) and the inventors tested the impact of ILA on T-cell polarization in vitro using the same polarizing cytokine conditions as above but replacing fecal water with ILA alone (1 mM). The inventors found a number of mRNA-transcripts induced, and these differed between Th1 and Th0, Th2 and Th17 polarizing conditions. In the presence of ILA, Th0, Th2 and Th17 cells upregulated the chemokine receptor, CXCR3 often associated with Th1-cells and granzyme B (FIG. 5F).

Moreover, these cells all strongly upregulated the negative regulator of T-cell activation, LGALS1 (Galectin-1)(Th0, P=2.19e⁻⁴²; Th2, P=2.47e⁻²⁶⁹; Th17, P=7.62e⁻⁴¹) suggesting an additional pathway for regulating pathogenic Th2 and Th17 immune responses in newborns (FIG. 5F). Also, in culture experiment using fecal water from B. infantis EVC001 supplemented children, upregulation of Galectin-1 was seen (FIG. 5D), further suggesting that ILA-mediatedsignaling explains some of the effects of B. infantis EVC001 supplementation in breastfed infants. In an animal model of zymosan-induced peritonitis, Galectin-1 has been reported to induce IL-27 and IL-10 and act through IFNβ-dependent reprogramming of tissue macrophages and be essential to resolve inflammation (Yaseen et al., 2020). The findings of HMO metabolizing microbes and the induction of tolerogenic responses are concurrent and associated with elevated IL-27 and IFNβ and ILA-mediated upregulation of Galectin-1 on CD4⁺ T-cells.

Taken together, the results indicate that during the first weeks of life there are transient immune responses to colonizing microbes, centered on mucosal surfaces. The colonization of the gut microbiome plays an integral role in this process and is likely, itself influenced by this process as well as other important determinants of health such as antibiotic use and breastfeeding. Specific bacteria, particularly those expressing HMO utilization genes, have nutritional advantages in breastfed infants and influence immune-microbe interactions by dampening inflammatory responses, in particular Th2 and Th17-type responses in favor of Th1 and regulatory T-cells. Key metabolites such as indole-3-lactic acid exert direct regulatory effects on Th2 and Th17 cells such as induction of regulatory Galectin-1, known to limit T-cell activation and collectively these layered effects of beneficial microbes and their metabolites on the developing immune system early in life have potential long-term consequences on the risk of developing immune-mediated diseases.

Summary Immune-microbe interactions early in life influence the risk of allergies, asthma and other inflammatory diseases. Breastfeeding guides healthier immune-microbe relationships by providing nutrients to specialized microbes that in turn benefit the host's immune system. Such bacteria have co-evolved with humans but are now increasingly rare in modern societies. Here the inventors show that a lack of bifidobacteria, and in particular depletion of genes required for HMO utilization from the metagenome is associated with systemic inflammation and immune dysregulation early in life. In breastfed infants given Bifidobacterium infantis EVC001, which expresses all HMO utilization genes, intestinal Th2 and Th17 cytokines were silenced and IFNβ induced. Fecal water from EVC001 supplemented infants contains abundant indole lactate, and B. infantis-derived indole-3-lactic acid (ILA) upregulated immunoregulatory Galectin-1 in Th2 and Th17 cells during polarization, providing a functional link between beneficial microbes and immunoregulation during the first months of life. Mounting evidence indicate that the composition of the infant gut microbiome is critical to immunological development, particularly during the first three months of life when aberrations in gut microbial composition are most influential in impacting the developing immune system.

The inventors now understand that the intestinal microbiome composition plays a critical role in the development of the immune system and influences an individual's risk of developing allergies, asthma, and some autoimmune disorders. Additionally, it is understood that human milk helps guide the development of healthy immune-microbe relationship, in part by providing the nutrients to specialized microbes that, in turn, benefit the host and its developing immune system. Importantly, bifidobacteria, which have co-evolved with humans, are associated with reduced risks of developing immune-mediated diseases but unfortunately are increasingly rare in modern societies; therefore, the inventors hypothesized that Bifidobacterium abundance may provide an evolutionary advantage to immunological sequences early in life. Moreover, utilizing a strain of bacteria that readily colonizes breastfed infants, the inventors evaluated fecal waters from infants colonized with B. infantis EVC001 and its major metabolites impact on CD4+ T cell polarization.

It is increasingly clear that early-life immune microbe interactions influence the risk of immune-mediated diseases later in life; however, the exact mechanisms remain elusive. Results herein extend the previous understanding of an immunological sequence of events, triggered by microbial colonization that results either in a balanced immune-microbe relationship or varying degrees of intestinal and systemic inflammation and perturbed immune cell regulation, most notable within the T-cell compartment. In particular, lack of bifidobacterial species and other microbes expressing HMO utilization genes, the inventors show to be associated with intestinal inflammation, driven by aberrant Th2 and Th17 responses. Using fecal water from infants colonized with B. infantis EVC001, polarized naïve T-cells towards Th1 in vitro, while fecal waters from infants not colonized with B. infantis EVC001 polarized Th2 and Th17 phenotype cells.

Tolerance induction to the microbiota is key to prevent tissue damage and inflammation and here the inventors make observations that indicate how such tolerance could be induced in human infants. The inventors find that Bifidobacteriaceae able to metabolize HMOs are associated with reduced pro-inflammatory markers and conversely elevated proteins such as IL-10 and IL-27, associated with regulatory T-cells. Also, memory Treg frequency was inversely correlated with proinflammatory monocyte abundance and activated T-cell population abundances in children with abundant Bifidobacteria, a regulatory relationship that is lost in children lacking such beneficial microbes. Moreover, the inventors uncover an additional possible inducer of tolerance, namely intestinal IFNβ which was much induced in infants fed B. infantis EVC001. IFNβ therapy in patients with multiple sclerosis induces IL-10 production by regulatory T-cells (Byrnes et al., 2002), and in mice IFNβ induce regulatory T-cells (Dikopoulos et al., 2005). The fact that systemic perturbations are still visible is a testament to the wide-ranging effects of immune-microbe interactions at mucosal surfaces on host physiology. The fact that mucosal-specific immune cells are circulating in the blood is known in celiac disease (Han et al., 2013), and IBD (Gorfu et al., 2009) patients. Although the B. infantis EVC001 strain is not detectable in both cohorts studied, the focus on HMO utilization genes allows for direct comparisons across these children. Importantly, the similarities in immune system states in relation to HMO utilization gene abundance serves as independent confirmation of the findings. It is also intriguing as this suggests that the imprinting effect on developing infant immune systems is not reliant on specific strains of microbes, but core functional properties of the metagenome, such as the ability to metabolize HMOs and produce key downstream metabolites such as indole-3-lactic acid. Organic acids, acetate and propionate have previously been implicated in mitigating lung inflammation in animal models (Trompette et al., 2014) and food allergy in human infants (Sandin et al., 2009). Indole-3-lactic acid, produced in breastfed infants colonized with B. infantis, has been shown to decrease enteric inflammation through activation of AhR and Nrf2 although the immune system changes were not resolved (Ehrlich et al., 2018; Meng et al., 2020). Further supporting the role of bifidobacteria-derived Indole-3-lactic acid is a recent report showing induced IL-22 production in CD4⁺ T cells and modulation of monocytes TNFα responses upon LPS-stimulation through AhR and hydrocarboxylic acid receptor 3 (Laursen et al., 2020). The data are in line with this study but adds more immunological details, such as the ILA-mediated direct effects on Th2 and Th17 cells and the upregulation of a negative regulator Galectin-1. This finding is also interesting in relation to data in patients with celiac disease, where substantial upregulation of Galectin-1 has been shown to induce tolerogenic intestinal responses (Sundblad et al., 2018). The inventors believe that this data indicates a mechanism of ensuring intestinal tolerance early in life, here shown to be induced by a specific bacterial metabolite.

Indeed, the correlation of HMO-utilization genes, specifically H5 gene abundance and the decrease in Th2-related cytokines with increased IL-27 is important given recent findings by Duar et al, 2020 that H5 is a key ecological determinant of fitness for Bifidobacterium species in the infant gut (Duar et al., 2020a) and this fitness advantage is likely both metabolic and dependent on the induction of immunological tolerance.

### Methods

Born-Immune newborn cohort study. The study was performed in accordance with the declaration of Helsinki and the study protocol was approved by the regional ethical board in Stockholm, Sweden (DNR: 2009/2052-31/3, 2014/921-32 and 2016/512-31/1). After obtaining informed consent from parents, blood samples from newborns and parents were collected at the Karolinska University Hospital. The inventors also collected fecal samples from infants, either at the time of clinical visits and frozen directly at −80 C or collected at home frozen at −20 C and brought to the clinic by parents. Clinical metadata such as mode of delivery, nutrition, growth and medications were gathered in a clinical database.

Blood immune cell profiling by Mass cytometry. Blood samples drawn from newborns and parents were mixed with a stabilizer (Brodin et al., 2019) (one of the components of Whole blood processing kit; Cytodelics AB, Sweden) either immediately or within 1-3 hours post blood draw and cryopreserved as per the manufacturer's recommendations. Samples were thawed, and cells were fixed/RBCs lysed using WASH #1 and WASH #2 buffers (Whole blood processing kit; Cytodelics AB, Sweden) as per the manufacturer's recommendations. This was performed a few days prior to barcoding and staining of cells. Post fix/lysis of cells, ˜1-2×10⁶ cells/sample were plated onto a 96 well round bottom plate using standard cryoprotective solution (10% DMSO and 90% FBS) and cryopreserved at −80° C. At the time of experimentation, cells were thawed at 37° C. using RPMI medium supplemented with 10% fetal bovine serum (FBS), 1% penicillin-streptomycin and benzonase (Sigma-Aldrich, Sweden). Briefly, cells were barcoded using automated liquid handling robotic system (Agilent technologies)(Mikes et al., 2019) using the Cell-ID 20-plex Barcoding kit (Fluidigm Inc.) as per the manufacturer's recommendations. Samples were pooled batch wise by keeping together the longitudinal samples from each newborn baby or parent in the same batch. Cells were then washed, FcR blocked for 12 min at room temperature, following which cells were incubated for another 30 min at 4° C. after addition of a cocktail of metal conjugated antibodies targeting the surface antigens. Cells were washed twice with CyFACS buffer (PBS with 0.1% BSA, 0.05% sodium azide and 2 mM EDTA) and fixed overnight using 2% formaldehyde made in PBS (VWR, Sweden). The broad extended panel of antibodies used are listed in Supplementary Table 1. For acquisition by CyTOF, cells were stained with DNA intercalator (0.125 μM Iridium-191/193 or MaxPar® Intercalator-Ir, Fluidigm) in 2% formaldehyde made in PBS for 20 min at room temperature. Cells were washed twice with CyFACS buffer, once with PBS and twice with milliQ water. Cells were mixed with 0.1× Norm Beads (EQ™ Four Element Calibration Beads, Fluidigm) filtered through a 35 μm nylon mesh and diluted to 1000,000 cells/ml. Samples were acquired using super samplers connected to our CyTOF2 mass cytometers (Fluidigm Inc.) using CyTOF software version 6.0.626 with noise reduction, a lower convolution threshold of 200, event length limits of 10-150 pushes, a sigma value of 3, and flow rate of 0.045 ml/min.

Antibodies and reagents for mass cytometry. The panel of monoclonal antibodies used for this study are indicated in the Key Resources Table. Monoclonal antibodies were either purchased pre-conjugated from Fluidigm or obtained in carrier/protein-free buffer as purified antibodies that were then coupled to lanthanide metals using the MaxPar X8 polymer conjugation kit (Fluidigm Inc.) as per the manufacturer's recommendations. Following the protein concentration determination by measurement of absorbance at 280 nm on a nanodrop, the metal-labeled antibodies were diluted in Candor PBS Antibody Stabilization solution (Candor Bioscience, Germany) for long-term storage at 4° C.

Born-immune plasma protein profiling. Plasma protein data was generated using Olink assays, a proximity extension assay (Olink AB, Uppsala)(Lundberg et al., 2011) For analysis, 20 μL of plasma from each sample was thawed and sent for analysis, either at the plasma protein profiling platform, Science for Life Laboratory, Stockholm or Olink AB in Uppsala. In these assays, plasma proteins are dually recognized by pairs of antibodies coupled to a cDNA-strand that ligates when brought into proximity by its target, extended by a polymerase and detected using a Biomark HD 96.96 dynamic PCR array (Fluidigm Inc.). Four Olink panels (CVD 2, CVD 3, Inflammation and Immune response) have been used as indicated in Key Resources Table, capturing a total of 355 unique proteins in each plasma sample.

Born-immune fecal metagenomics. DNA was extracted as in IHMS DNA extraction protocol #8 (Costea et al., 2017). According to protocol recommendations 0.2 g of faeces were used. Briefly, samples were treated with lysozyme solution and subjected to bead beating using zirconium beads. After centrifugation, DNA is extracted from supernatants with QIAamp Fast DNA Stool Mini Kit (Qiagen, Cat No. 51604). After DNA extraction, collected DNA was quantified with Qubit (ThermoFisher, Cat No. Q32851) and 10 ng were subjected to mechanical fragmentation with the Covaris Focused-ultrasonicator to ensure that fragment sizes were compatible with Illumina sequencing (˜300 bp average). Sequencing adapters and sample barcodes were incorporated to the DNA fragments using ThruPLEX DNA-seq kit (Rubicon Genomics, Cat No. R400406). ThruPLEX DNA-seq products were purified and size selected by AMPure beads (Beckman Coulter, Cat No. B23318), and DNA concentration and size distribution were inspected with the Qubit dsDNA HSAssay Kit (ThermoFisher, Cat No. Q32851) and the Agilent 2100 Bioanalyzer High Sensitivity kit (Agilent Technologies, Cat No. 5067-4626), respectively. Purified ThruPLEX DNA-seq products were then equimolarly pooled in 4 lanes and subjected to NovaSeq 6000 S4 IlluminaSequencing at the Science for Life Laboratory, Stockholm, Sweden.

T-cell polarization experiments. PBMC derived CD3 CD4 CD45RA″ naïve T-cells (CD8/14/19/56 negative) into an enriched culture medium including RPMI 1640+10% FBS+NEAA+1% Pen-strep+55 μM β-mercaptoethanol. Cells are added at a concentration of 2×10⁵ cells/ml and pre-washed and resuspended 2.5 ml T-Activator Dynabeads are added to obtain a bead:cell ratio of 1:2. One milliliter of bead/cell suspension was added to 24-well plate and the following polarizing supplements Th0: No cytokines, Th1: IL-12 (50 nanogram (ng)/ml), Anti-human IL-4 antibody (1 μg/ml) Th2: IL-4 (10 ng/ml), Anti-human IFN-γ antibody (1 μg/ml), Th17: IL-6 (50 ng/ml), IL-23 (20 ng/ml), IL-1B (10 ng/ml), TGF-β1 (5 ng/ml), Anti-hum IL-4 (1 μg/ml), Anti-human IFN-γ antibody (1 μg/ml), iTreg: TGF-β1 (5 ng/ml), IL-2 (10 ng/ml) IFN-β: IFN-β (10 ng/ml). Cells are incubated at 37º C, 5% CO₂ humidified incubator for 5 days and harvested.

Targeted transcriptome and protein by BD Rhapsody single cell RNA Sequencing. Polarized T cells in each condition were labeled using BD Single-Cell Multiplexing Kit and BD AbSeq Ab-Oligos reagents strictly following the manufacturers protocol (BD Biosciences). Briefly, cells from each experiment condition were labelled with each sample tag and pooled 18 AbSeqAb-Oligos. Each sample was then washed twice, counted and resuspended in cold BD SampleBuffer, then calculated the cell number in each sample, then pooled the required number of cells from each sample to get approximately 20,000 cells in 620 μl for each cartridge (around 9 samples for each cartridge). After priming the nanowell cartridges, the pooled sample was loaded onto BDRhapsody cartridges and incubated at room temperature. Cell Capture Beads were prepared and then loaded onto the cartridge. According to the manufacturers protocol, cartridges were washed, cells were lysed, and Cell Capture Beads were retrieved and washed prior to performing reverse transcription and treatment with Exonuclease I. cDNA Libraries were prepared using mRNA Targeted, Sample Tag, and BD AbSeq Library Preparation with the BD Rhapsody Targeted mRNA and AbSeq Amplification Kits and protocol. In brief, cDNA targeted amplification using the Human T cell Expression Panel primers via PCR. mRNA PCR products were separated from sample tag and AbSeq products with double-sided size selection using AMPure XP magnetic beads (Beckman Coulter). mRNA and Sample Tag products were further amplified using PCR. PCR products were then purified using AMPure XP magnetic beads. Quality and quantity of PCR products were determined by using an Agilent 2100 Bioanalyzer and Qubit Fluorometer using the Qubit dsDNA HS Kit (ThermoFisher). Targeted mRNA product was diluted to 2.5 ng/μL and sample tag and AbSeq PCR products were diluted to 1 ng/μL to prepare final libraries. Final libraries were indexed using PCR. Index PCR products were purified using AMPure XP magnetic beads. Quality of final libraries was assessed by using Agilent Bioanalyzer and quantified using a Qubit Fluorometer. Final libraries were diluted to 2 nM for paired-end (150 bp) sequencing on a NovaSeq sequencer (Illumina).

Olink Preprocessing. Plasma protein data was batch corrected and normalized on the basis of NPX values across batches with available bridge samples.

Mass cytometry Preprocessing. All FCS-files unrandomized using CyTOF software (version 6.0.626) were transferred with no further preprocessing. An automated cell classification supervised algorithm, Grid was used to manually define reference subpopulations and then train a learning algorithm (XGBoost) to recognize the same subsets of cells in novel data, providing a rapid and robust cell classification method for high-dimensional Mass cytometry datasets (Chen et al., 2020). FCS files and relevant phenotypic markers were used in order to manually gate cell populations to be used as a reference, then the reference was used to train a classifier algorithm to categorize similar cells. The output results in a dataframe with samples as columns and cell sub-populations as rows. Outputs from all Mass cytometry experiments were merged, and batch differences removed using limma (Ritchie et al., 2015).

Metagenome data-quality filtering and host removal. 347 and 60 demultiplexed fastq files from the Born-immune and IMPRINT cohort respectively were downstream processed using the same pipeline and parameters. Demultiplexed samples were quality filtered using fastp v0.20.0 (Chen et al., 2018), and host contamination removed using Kraken v2.0.8_beta (Wood et al., 2019) by mapping against the NCBI's GRCh38.p13 database. Both steps were run using default settings in StaG-mwc v.0.4.1 (doi.org/10.5281/zenodo.1483891).

Metagenome data—Taxonomic and functional profiling. Taxonomic profiles were established using MetaPhlAn v3.0.5 (Beghini et al., 2020) and functional HMO profiles generated with HUMAnN2 v.2.8.1 (Franzosa et al., 2018) bypassing all steps except “nucleotide-search” and “evalue 0.00001” with a customized nucleotide database of HMO genes instead of the chochophlan database. RPKs from HUMAnN2 were normalized to cpms using ‘humann2_renorm_table’. Both taxonomic and functional profiling were incorporated into StaG-mwc.

Bifidobacteria abundance correlation. Immune cell and plasma protein data was reduced to include infant samples collected 56 to 152 days after birth with correlating metagenomics data, resulting in n=18 and n=19 samples respectively. Fold change was calculated, and variables re-ordered in descending value order. The correlation matrices were built using library corrplot with spearman method, for comparison purposes low bifidobacterial matrix was reordered to the level order of high bifidobacterial matrix.

HMO correlation. Samples were binned according to days after birth while HMO utilization genes were clustered in accordance with pathway and function. The CPM counts were binned as well with increasing ranges and heatmap was built using library superheat. Correlations with individual cytokines were based on NPX values and CPM counts. ANOVA test was performed for Spearman correlation performed between individual cytokines and HMO utilization genes.

Targeted transcriptomics processing. FASTQ files of targeted transcriptomics data were processed on the Seven Bridges platform using the Targeted Analysis Pipeline v1.9 (BD Biosciences)(www.sevenbridges.com). R1 and R2 are filtered removing low quality sequencing reads, checking read lengths as well as lengths of strings of identical bases. Read pair is removed if read length of R1 is less than 66 bases or R2 is less than 64 bases. R1 reads are annotated to cell label sequences and unique molecular identifiers (UMI), perfect matches are kept while others will be held for further filtering. R2 reads are annotated to oligo sequence to genes on targeted panel by Bowtie2. Then, all valid R1 and R2 read pairs are collapsed into unique raw molecules. For all analysis, the inventors used output of distribution-based error correction (DBEC) as a means to correct for both artefacts in PCR cycles and sequencing errors. Expression matrices containing DBEC-adjusted molecule counts after sample tag assignment were used for downstream analysis.

Analysis of Seurat object with targeted data. The expression matrices were read into the R package Seurat v3 (Butler et al., 2018) where they were merged and split between RNA and antibody (Ab) assays thereafter using scripts from github.com/MairFlo/Targeted_transcriptomics (Mair et al., 2020). After creating Seurat object that included features that were detected in at least 3 cells and cells that were detected in at least 50 features within the RNA assay. In the first experiment relating to Bifidobacterium, out of 53,184 cells, 2,911 were called as multiples and 877 events as undetermined. Multiples and undetermined cells were removed from the analysis. In the second experiment relating to ILA, out of 42, 151 cells, 5,539 were called as multiples and 17 events as undetermined. On RNA assay, a natural log normalization was performed with a scale factor of 10,000 while a centered log-ratio normalization was performed on the Ab assay. Both assays were linearly scaled to remove uninteresting sources of variation like batch effect. Additionally, RNA assay was scaled to regress out the total number of molecules identified within a cell as well as the effect of GAPDH gene. The effect of the GAPDH gene was regressed out by computing the fraction of counts from that gene. All genes or proteins were used for dimensionality reduction using UMAP and clustering.

Partition-based graph abstraction of single-cell data. Partition-based graph abstraction (PAGA) (Wolf et al., 2019) was utilized to demonstrate the topology abstraction of single-cell RNA data. In brief, PCA was first applied to reduce the dimension of RNA data to 20, and then a kNN-like graph was built with the approximate nearest neighbor search. Afterwards, the highly connected communities in the kNN-like graph were discovered with Leiden method (Traag et al., 2019), which were further utilized by PAGA to infer a trajectory map, which demonstrates the topology relationship of those highly connected communities. Finally, the trajectory map was used as the initial position and the scatters of single cells were embedded with ForceAtlas2 (Jacomy et al., 2014) for visualization.

mRNA-seq data analysis. Quality control for the bulk RNA-sequencing of FACS-sorted immune cell populations was provided by the National Genomics Infrastructure (NGI) at Science for Life Laboratory, Stockholm, Sweden. First, we quantified abundances of transcript sequences in FASTA format by generating abundance estimates for all samples using the Kallisto software (Bray et al., 2016). Also, gene abundance estimates were performed by summing the transcript expression (TPM) values for the transcripts of the same gene. Since DESeq2 expects count data from the Kallisto output the tximport package was used to convert these estimates into read counts. DESeq2 was performed as a basis for differential gene expression analysis based on the negative binomial distribution (Love et al., 2014). The inventors employed a design to demonstrate differential gene expression between circulating CD38−CD4−CD62Lneg and memory CD4T cells over time. Low gene counts (<100) were filtered out and variance stabilizing transformation (VST) was performed on the count data.

Gene Set Enrichment Analysis (GSEA). Gene set enrichment analysis (GSEA) was performed to identify transcriptomic differences occurring over time in circulating CD38⁺CD62L⁻ memory CD4⁺ T cells vs. Total memory CD4⁺T-cells. The R package fgsea was used to find the most highly enriched hallmark pathways through gmtPathways function (Subramanian et al., 2005). Pathways of interest were subsequently isolated and all genes within each pathway observed using volcano plots. Details of the study design and procedures used to collect these samples has been reported elsewhere (Frese et al., 2017; Smilowitz et al., 2017). Briefly, exclusively breastfed term infants were randomly selected to receive 1.8×10¹⁰ colony-forming units (CFU) activated B. infantis EVC001 daily for 21 days (EVC001) starting at day 7 postnatal or to receive breast milk alone (control) and followed up to postnatal day 60 (Frese et al., 2017). All mothers received lactation support throughout the study. The demographic information (e.g., age, sex, and gestational age) was collected from each participant. All aspects of the study were approved by the University of California Davis Institutional Review Board (IRB Number: ID 631099) and all participants provided written informed consent. Here fecal samples from individual subjects were chosen at random and made up a subset of the original study participants. Fecal samples from randomly selected infants who were fed EVC001 (n=20) and control infants (n=20) on days 6 (Baseline) and 60 postnatal were collected and analyzed for fecal enteric cytokine concentrations (described below). Day 21 fecal samples were used for non-targeted fecal metabolomics analysis (described below). Day 21 metagenomics, which have been reported elsewhere (Casaburi and Frese, 2018) were used for correlative analyses. All sequencing libraries generated in this study have been deposited with the NCBI SRA (PRJNA390646) and are publicly available.

Fecal microbiome analyses. Sequences of DNA previously extracted from approximately 100 mg of frozen stools collected from infants on day 21 postnatal (Frese et al., 2017) were determined. Briefly, the DNA was subjected to bead beating prior to column purification using a Zymo Fecal DNA Miniprep kit, according to the manufacturer's instructions. Metagenomic shotgun library preparation and sequencing was performed at the California Institute for Quantitative Biosciences (QB3) (University of California, Berkeley) on an Illumina HiSeq 4000 platform using a paired-end sequencing approach with a targeted read length of 150 bp and an insert size of 150 bp (Casaburi et al., 2019). Demultiplexed fastq sequences were quality filtered, including adaptor trimming using Trimmomatic v0.36 (Bolger et al., 2014) with default parameters. Quality-filtered sequences were screened to remove human sequences using GenCoF v1.0 (Czajkowski et al., 2018) against a non-redundant version of the Genome Reference Consortium Human Build 38, patch release 7 (GRCh38_p7; www.ncbi.nlm.nih.gov). Human sequence-filtered raw reads were deposited in the Sequence Read Archive (SRA; www.ncbi.nlm.nih.gov/sra) under the reference number, PRJNA390646. Taxonomic profiling of the metagenomic samples was performed using MetaPhlAn2 (Truong et al., 2015), which uses a library of clade-specific markers to provide pan-microbial (bacterial, archaeal, viral, and eukaryotic) profiling (huttenhower.sph.harvard.edu/metaphlan2). Strain characterization was performed using PanPhlan (Scholz et al., 2016) which is used in combination with MetaPhlAn2 to characterize strain-level variants in marker genes for a selected organism. For PanPhlan analysis, the pangenomes from Bifidobacterium longum (bitbucket.org/CibioCM/panphlan) were used as a reference. Both MetaPhlAn2 and PanPhlan were used with their default settings as described in the updated global profiling of the Human Microbiome Project (2017)(Lloyd-Price et al., 2017).

Additionally, 16S rRNA libraries from Day 6 and Day 60 postnatal were generated and sequenced (Frese et al., 2017). Briefly, the V4 region of the 16S rRNA gene was amplified and sequenced using primers 515f and 806r as previously described with recent modifications (Caporaso et al., 2011; Walters et al., 2016). Paired-end DNA (300 bp) sequencing was performed at the UC Davis Genome Center on an Illumina MiSeq system. Sequences were analyzed using QIIME 1.9.1 (Caporaso et al., 2010). Open-reference operational taxonomical unit (OTU) picking was performed using UCLUST at 97% identity (Edgar, 2010). OTUs with a relative abundance of less than 0.005% were removed (Bokulich et al., 2013). Quality filtering and removal of human sequences from shotgun metagenomes

Absolute quantification of B. infantis by Quantitative Real-Time PCR. As previously described in Frese et al 2017, quantification of the total B. infantis was performed by quantitative real-time PCR using Blon_0915 primers Blon0915F (5′-CGTATTGGCTTTGTACGCATTT-3′), Blon0915R (5′-ATCGTGCCGGTGAGATTTAC-3′) and BI915 PRB (5′-6-FAM-CCAGTATGG-ZEN-CTGGTAAAGTTCACTGCA-3IABkFQ). Each reaction contained 10 μL of 2× TaqMan Universal Master Mix II with UNG master mix (Applied Biosystems), 0.9 μm of each primer, 0.25 μM probe and 5 μL of template DNA. Thermal cycling was performed on a QuantStudio 3 Real-Time PCR System and consisted of an initial UNG activation step of 2 minute at 50° ° C. followed by a 10-minute denaturation at 95° C. succeeded by 40 cycles of 15 s at 95° C. and 1 min at 60° C. Quantitative PCR was carried out using standard curves of known B. infantis EVC001 cultures prepared by serial dilution. All samples including the standard curve were ran in duplicate.

Fecal cytokine analysis. Interleukin (IL)-4, IL-12p70, IL-13, IL-17A, IL-21, IL-23, IL-27, IL-31, IL-33, IFN, and MIP3a were quantified from 80 mg of stool diluted 1:10 in Meso Scale Discovery (MSD; Rockville, MD) diluent using the U-PLEX Inflammation Panel 1 (human) Kit according to the manufacturer's instructions. Standards and samples were measured in duplicate and blank values were subtracted from all readings. Assays were performed at least twice. All detectable biomarker values were included as continuous data in the analyses; however, values below level of detection (<20% of all cytokines measurements) were generated below the level of quantification to justify parametric statistics. Fecal cytokine concentrations were determined using calibration curves to which electrochemiluminescence signals were backfitted. Final concentrations were calculated using the Sector Imager 2400 MSD Discovery Workbench analysis software, as was previously published (Henrick et al., 2019; Nguyen et al., 2021).

Fecal water preparation. Historical fecal samples from term infants either colonized with B. infantis EVC001 or not were collected and stored in −80° C. until processing. Pooled fecal samples (minimum of 3) from infants colonized with B. infantis EVC001 or not were weighed and diluted 25% w/v in sterile PBS and vortexed for 1 min allowing stool to thaw and form a homogenous slurry. Fecal slurries were then centrifuged for 30 min at 4,000 RPM at 4° C. The supernatant was collected and spun again for 3 hours at 12,000 RPM at 4° C. The supernatant was further collected and then serially filtered (40 μm cell strainer, 1 um, 0.45 um, and 0.22 um). Filtered fecal waters were stored in −80° ° C. until use.

Fecal metabolomics analysis. Fecal samples were sent to Metabolon, Inc. (Durham, NC) for non-targeted metabolite profiling. Forty fecal samples, control (n=20) and B. infantis EVC001-fed (n=20) from day 21 postnatal were collected and processed for non-targeted metabolomics profiling, as shown previously (Call et al., 2018). Briefly, samples were exposed to a combination of aqueous and organic solvents to extract small molecules. Residual organic solvent was removed using a TurboVap (Zymark), and the fecal extracts were lyophilized and divided equally for then equal GC/MS and UPLC-MS/MS analysis in parallel. Extracts were derivatized with bistrimethyl-silyl-triflouroacetamide and analyzed using a Trace DSQ (Thermo-Finnigan) mass spectrometer Fecal extracts were analyzed under both acidic and basic conditions using an ACQUITY (Waters) UPLC and an LTQ (Thermo-Finnigan) mass spectrometer. Fecal compounds were identified by comparison of the raw data with Metabolon's curated library of standards. The values for compounds in the fecal samples were normalized by the dry mass of the sample and missing values were imputed with half the compound minimum. Absolute compound intensity values were used to calculate fold differences between controls and EVC001-fed samples, while for all other analyses, the values were transformed using the generalized log transformation then mean-centered and scaled by the standard deviation.

Statistical Analysis. All statistical analyses were performed in R v3.6.2. A Kruskal-Wallis one-way analysis of variance coupled with an FDR or Bonferroni correction was used for statistical comparisons between individual genes, cytokines and taxa amongst groups. Statistical analysis to assess total resistome or enterotype composition by group was performed using a Mann-Whitney or Holm-adjusted Dunn's test. Rarefaction curves were computed to estimate the diversity of the identified ARGs across samples. A nonparametric two-sample t-test was used to compare rarefaction curves using Monte Carlo permutations (n=999). Enterotype analysis was performed as previously described (Arumugam et al., 2011). Cytokines and B. infantis-specific qPCR were correlated with the Spearman method with FDR correction. The P-values throughout the manuscript are represented by asterisks (*, P<0.05; **, P<0.01; ***, P<0.001; ****, P<0.0001).

Data and code availability. Raw and processed data is available for download from in our Mendeley Datarepository: doi:10.17632/gc4d9h4×67.1. Scripts for recreating figures in the paper: github.com/rodriluc/Bifido_newborns/

TABLE 1
Antibodies used in mass cytometry analysis

| | | |
|---|---|---|
| REAGENT or RESOURCE | SOURCE | IDENTIFIER |

Mass cytometry - Broad extended panel

| | | |
|---|---|---|
| Anti-human CD3e (UCHT1), Sm-154 | Fluidigm | Cat# 317302; RRID: AB_571927 |
| Anti-human CD4 (RPA-T4), Purified | Biolegend | Cat# 300502; RRID: AB_314070 |
| Anti-human CD5 (UCHT2), Purified | Biolegend | Cat# 300602; RRID: AB_314088 |
| Anti-human CD7 (CD7-6B7), Purified | Biolegend | Cat# 343102; RRID: AB_1659214 |
| Anti-human CD8 (SK1), Purified | Biolegend | Cat# 344702; RRID: AB_1877104 |
| Anti-human CD9 (SN4 C3-3A2), Purified | eBiosciences | Cat# 14-0098-82; RRID: AB_657777 |
| Anti-human CD11B (Mac-1) - 209Bi | Fluidigm | Cat# 3209003B; RRID: AB_2687654 |
| Anti-human CD11c (Bu15), Purified | Biolegend | Cat# 337202; RRID: AB_1236381 |
| Anti-human CD14 (M5E2), Purified | Biolegend | Cat# 301802; RRID: AB_314184 |
| Anti-human CD15 (W6D3), Purified | Biolegend | Cat# 323002; RRID: AB_756008 |
| Anti-human CD16 (3G8), Bi-209 | Fluidigm | Cat# 3209002B, RRID: AB_2756431 |
| Anti-human CD19 (HIB19), Nd-142 | Fluidigm | Cat# 3142001B; RRID: AB_2651155 |
| Anti-human CD20 (2H7), Purified | Biolegend | Cat# 302302; RRID: AB_314250 |
| Anti-human CD22 (HIB22), Purified | Biolegend | Cat# 302502; RRID: AB_314264 |
| Anti-human CD24 (ML5), Purified | Biolegend | Cat# 311102; RRID: AB_314851 |
| Anti-human CD25 (2A3), Sm-149 | Fluidigm | Cat# 3149010B, RRID: AB_2756416 |
| Anti-human CD26 (BA5b), Purified | Biolegend | Cat# 302702; RRID: AB_314286 |
| Anti-human CD27 (L128), Er-167 | Fluidigm | Cat# 3167006B; RRID: N/A |
| Anti-human CD29 (TS2/16), Purified | Biolegend | Cat# 303002; RRID: AB_314318 |
| Anti-human CD31 (WM59) - Purified | BioLegend | Cat# 303102; RRID: AB_314328 |
| Anti-human CD33 (WM53), Purified | Biolegend | Cat# 303402; RRID: AB_314346 |
| Anti-human CD34 (581), Purified | Biolegend | Cat# 343502; RRID: AB_1731898 |
| Anti-human CD38 (HIT2), Purified | Biolegend | Cat# 303502; RRID: AB_314354 |
| Anti-human CD39 (A1), Purified | Biolegend | Cat# 328202; RRID: AB_940438 |
| Anti-human CD44 (BJ18) - Purified | BioLegend | Cat# 338802; RRID: AB_1501199 |
| Anti-human CD45 (HI30), Y-89 | Fluidigm | Cat# 3089003B; RRID: AB_2661851 |
| Anti-human CD45RA (HI100), Tm-169 | Fluidigm | Cat# 3169008B; RRID: N/A |
| Anti-human CD49d (9F10), Pr-141 | Fluidigm | Cat# 3141004B; RRID: N/A |
| Anti-human CD56 (NCAM16.2), Purified | BD | Cat# 559043; RRID: AB_397180 |
| Anti-human CD57 (HCD57), Purified | Biolegend | Cat# 322302; RRID: AB_535988 |
| Anti-human CD64 (10.1), Purified | Biolegend | Cat# 305002, RRID: AB_314486 |
| Anti-human CD99 (HCD99), Purified | Biolegend | Cat# 318002; RRID: AB_604112 |
| Anti-human CD123 (6H6), Purified | Biolegend | Cat# 306002; RRID: AB_314576 |
| Anti-human CD127 (A019D5), Ho-165 | Fluidigm | Cat# 3165008B; RRID: N/A |
| Anti-human CD161 (HP-3G10), Purified | Biolegend | Cat# 339902; RRID: AB_2661837 |
| Anti-human HLA-ABC (W6/32), Purified | Biolegend | Cat# 311402; RRID: AB_1076699 |
| Anti-human HLA-DR (L243), Purified | Biolegend | Cat# 307602; RRID: AB_314680 |
| Anti-human IgD (IA6-2), Purified | Biolegend | Cat# 348202; RRID: AB_10550095 |
| Anti-human Siglec-8 (837535), Purified | R&D Systems | Cat# MAB7975; RRID: N/A |
| Anti-human TCRgd (5A6.E9), Purified | Fischer Scientific | Cat# TCR1061; RRID: AB_223500 |

Biological Samples

| | | |
|---|---|---|
| Whole blood samples from newborns | Karolinska | N/A |
| andparents | UniversityHospital | |
| Fecal samples from newborn children | Karolinska | N/A |
| | UniversityHospital | |

Chemicals, Peptides, and Recombinant Proteins

| | | |
|---|---|---|
| Bovine Serum Albumin | Sigma-Aldrich | Cat# A3059; RRID: N/A |
| Cell-ID Intercalator-Ir | Fluidigm | Cat# 201192B; RRID: N/A |
| Cell-ID 20-Plex Pd Barcoding Kit | Fluidigm | Cat# 201060; RRID: N/A |
| DMSO | Sigma-Aldrich | Cat# D8418; RRID: N/A |
| EDTA | Rockland | Cat# MB-014; RRID: N/A |
| EQ Four Element Calibration Beads | Fluidigm | Cat# 201078; RRID: N/A |
| FBS | Sigma-Aldrich | Cat# 12103C; RRID: N/A |
| Fc Receptor (FcR) blocking buffer | Cytodelics | Customized |
| Maxpar Water | Fluidigm | Cat# 201069; RRID; N/A |
| Maxpar X8 Multimetal Labeling Kit (40 rxn) | Fluidigm | Cat# 201300; RRID; N/A |
| Metal isotopes as chloride salts (In-115, | Trace Sciences | Customized |
| Gd-155, Gd-157, Dy-161, Dy-163, Yb-173) | International | |
| Paraformaldehyde | VWR | Cat# 16005; RRID: N/A |
| Penicillin-streptomycin | Sigma-Aldrich | Cat# P4333; RRID: N/A |
| Protein Stabilizer PBS | Candor Bioscience | Cat# 131125, RRID: N/A |
| PBS 1X | Rockland | Cat# MB-008; RRID: N/A |
| RPMI 1640 medium | Sigma-Aldrich | Cat# R848; RRID: N/A |
| Sodium Azide | Sigma-Aldrich | Cat# 71289; RRID: N/A |
| Whole blood (human) processing kit | Cytodelics | Cat# hC001-500; RRID: N/A |

Critical Commercial Assays

| | | |
|---|---|---|
| Inflammation I panel | Olink AB | N/A |
| Cardiovascular (CVD) II panel | Olink AB | N/A |
| Cardiovascular (CVD) III panel | Olink AB | N/A |
| Immune Response panel | Olink AB | N/A |

Other

| | | |
|---|---|---|
| BenchBot robot | Agilent technologies | Customized |
| Bravo liquid handling platform | Agilent technologies | Customized |
| CyTOF 2 mass cytometer | Fluidigm | N/A |
| EL406 Washer Dispenser | BioTek | Customized |
| pluriStrainer Mini, 40 μm | pluriSelect | Cat# 43-10040-70; RRID: N/A |
| Polypropylene tubes | Sarstedt | Cat# 55526; RRID: N/A |
| TC20 automated cell counter | BioRad | N/A |
| Vspin microplate centrifuge | Agilent technologies | Customized |

Software and Algorithms

| | | |
|---|---|---|
| Seven Bridges Platform | — | https://www.sevenbridges.com/platform/ |
| R 4.0.2 | R Core Team, 2020 | https://www.r-project.org/ |
| Python 3.7 | | https://www.python.org/ |
| Seurat | | https://cran.r-project.org/web/packages/Seurat/index.html |
| DESeq2 | Love et al., 2014 | https://bioconductor.org/packages/release/bioc/html/DESeq2.html |
| tmod 0.46.2 | | https://cran.r-project.org/package=tmod |
| kallisto | Bray et al., 2016 | https://github.com/pachterlab/kallisto |
| fgsea | Subramanian et | http://bioconductor.org/packages/release/bioc/html/fgsea.html |
| | al., 2005 | |
| Scanpy | Wolf et al., 2018 | https://github.com/theislab/Scanpy |
| PAGA | Wolf et al., 2019 | https://github.com/theislab/paga |

BD Rhapsody antibodies and reagents

| | | |
|---|---|---|
| BD AbSeq Hu CCR7(CD197) | BD | Cat # 940394 |
| BD AbSeq Hu CD137 | BD | Cat # 940055 |
| BD AbSeq Hu CD28 | BD | Cat # 940017 |
| BD AbSeq Hu CD11a | BD | Cat # 940077 |
| BD AbSeq Hu TCR galma/delta | BD | Cat # 940057 |
| BD AbSeq Hu CD103 | BD | Cat # 940067 |
| BD AbSeq Hu CD27 | BD | Cat # 940018 |
| BD AbSeq Hu CD39 | BD | Cat # 940073 |
| BD AbSeq Hu CD62L | BD | Cat # 940041 |
| BD AbSeq Hu CD4 | BD | Cat # 940001 |
| BD AbSeq Hu CD8 | BD | Cat # 940003 |
| BD AbSeq Hu CD3 | BD | Cat # 940000 |
| BD AbSeq Hu CD16 | BD | Cat # 940006 |
| BD AbSeq Hu CD56 | BD | Cat # 940007 |
| BD AbSeq Hu CD161 | BD | Cat # 940070 |
| BD AbSeq Hu CD45RA | BD | Cat # 940011 |
| BD AbSeq Hu CD38 | BD | Cat # 940013 |
| BD AbSeq Hu HLA-DR | BD | Cat # 940010 |
| BD single cell multiplexing kit | BD | Cat # 633781 |
| BD Rhapsody cartridge reagent kit | BD | Cat # 633731 |
| BD Rhapsody cDNA kit | BD | Cat # 633773 |
| BD Rhapsody targeted mRNA and AbSeq | BD | Cat # 633774 |
| amplification kit | | |
| BD Pharmingen Stain Buffer (FBS) | BD | Cat # 554656 |
| DRAQ7 | BD | Cat # 564904 |
| BD Rhapsody Human T-Cell Expression | BD | Cat # 633751 |
| Panel | | |

Critical Commercial Assays

| | | |
|---|---|---|
| Zirconium beads 0.1 mm | Qiagen | 13118-400 |
| Lysozyme | ThermoFisher | 89833 |
| Proteinase K | Qiagen | 19133 |
| ThruPLEX ® DNA-seq 96D | Takara | R400676 |
| Agencourt AMPure XP | Beckmann Coulter | A63881 |
| Qubit ™ Assay Tubes, | ThermoFisher | Q32856 |
| Qubit ™ dsDNA HS Assay Kit | ThermoFisher | Q32854 |
| QIAamp Fast DNA Stool Mini Kit | Qiagen | 51604 |
| Agilent High Sensitivity DNA Kit | Agilent | 5067-4626 |
| 96 microTUBE Plate | Covaris | 520078 |

TABLE 2
Significance of fecal cytokine measurements

| | Day 6 (baseline) | | | | | Day 60 | | | | |
|---|---|---|---|---|---|---|---|---|---|---|
| | median (SD), pg/mg | | | | | median (SD), pg/mg | | | | |
| Cytokine | control | | EVC001-fed | | P - value | control | | EVC001-fed | | P - value |
| IL-4 | 0.07 | (0.03) | 0.07 | (0.24) | 0.08 | 14.1 | (44.03) | 6.88 | (5.83) | 0.11 |
| IL-12p70 | 0.24 | (0.19) | 0.22 | (0.48) | 0.74 | 0.16 | (0.26) | 0.12 | (26.85) | 0.21 |
| IL-13 | 3 | (5.31) | 3 | (12.27) | 0.33 | 366.62 | (333.26) | 247.56 | (103.62) | 0.015 |
| IL-17A | 7.85 | (11.75) | 8.89 | (24.15) | 0.45 | 177.95 | (266.16) | 19.78 | (88.43) | 0.0029 |
| IL-21 | 9.95 | (4.45) | 8.98 | (4.62) | 0.21 | 6.88 | (10.96) | 4.38 | (0.99) | 0.00066 |
| IL-23 | 3.45 | (3.01) | 2.56 | (3.58) | 0.21 | 2.16 | (4.22) | 1.55 | (134.79) | 0.16 |
| IL-27 | 42.14 | (20.91) | 34.41 | (23.03) | 0.16 | 21.58 | (24.81) | 17.67 | (17.65) | 0.11 |
| IL-31 | 12.73 | (9.76) | 12.98 | (9.77) | 1 | 14.45 | (3.33) | 10.64 | (3.26) | 0.007 |
| IL-33 | 0.81 | (0.26) | 0.67 | (0.28) | 0.45 | 0.48 | (0.25) | 0.26 | (0.07) | 0.00011 |
| MIP-3a | 1 | (0.54) | 0.95 | (0.46) | 0.48 | 0.93 | (0.91) | 0.58 | (0.24) | 0.0078 |
| IFN-B | 12.37 | (87.54) | 15.01 | (17.95) | 0.83 | 11.12 | (21.49) | 17.52 | (43.52) | 0.016 |

### Example 3. Fecal Waters with Different Microbiomes

Experiment to determine what is needed to polarize T cells into regulatory T cells\collection of fecal waters

Fecal Water Preparation. Fecal sample DNA is analysed for microbiome composition using qPCR, NGS, and/or shot-gun metagenomic sequencing. Fecal samples are diluted in sterile 37° C. PBS containing 20% FBS to a final concentration of 1 g/mL. Diluted fecal samples are then vortex for 1 minutes and incubated at 37° ° C. for 10 minutes. Following incubation, samples are centrifuged at ˜4303 g (max speed) for 10 minutes at room temperature in 50 mL conical tube. Supernatant is aliquoted into 2 mL centrifuge tubes and centrifuged at 14,000 rpm (max speed) for 2.5 hours in 4 C. Samples are serially filtered through 5 mm filter, 1 μm filter, 0.45 μm filter, and finally 0.22 μm filter to remove intact host and bacterial cells. The remaining liquid (fecal water) is aliquoted into 2 mL centrifuge tubes and stored at −80 C.

### Example 4. Characterization of Fecal Water with Different Underlying Microbiomes

Fecal samples are collected and processed for non-targeted metabolomics profiling, as shown previously (Call et al., 2018). Briefly, samples are exposed to a combination of aqueous and organic solvents to extract small molecules. Residual organic solvent is removed using a TurboVap (Zymark), and the fecal extracts are lyophilized and divided equally for GC/MS and UPLC-MS/MS analysis in parallel. Extracts are derivatized with bistrimethyl-silyltriflouroacetamide and analyzed using a Trace DSQ (Thermo-Finnigan) mass spectrometer. Fecal extracts are analyzed under both acidic and basic conditions using an ACQUITY (Waters) UPLC and an LTQ (Thermo-Finnigan) mass spectrometer. Fecal compounds are identified by comparison of the raw data to curated libraries of standards. The values for compounds in the fecal samples are normalized by the dry mass of the sample and missing values are imputed with half the compound minimum. Absolute compound intensity values are used to calculate fold differences between controls and EVC001-fed samples, while for all other analyses, the values are transformed using the generalized log transformation then mean-centered and scaled by the standard deviation. For quantification of specific metabolites, like ILA, acetate, or lactate, metabolite quantification can be completed using GC/MS and UPLC-MS/MS of samples with known concentrations of specific metabolites added in the run. Sample concentrations (i.e. acetate, lactate, indolelactates, bile salts, etc.). are determined using values run against known standard concentrations. In some cases, specific fecal water cytokines and bacterial metabolites can be assessed through ELISA-based analysis. Indolelactates are also a major bacterial metabolite produced ˜10-fold higher in B. infantis colonized infants compared to infants not colonized with B. infantis.

### Example 5. Metabolite Profile for Indole-3-Lactic-Acid from In Vitro Growth Profiling on Various Carbon Sources

In vitro bacterial growth. Supernatant from bacterial cultures grown on specific carbon sources was collected for metabolite profiling. All bacterial growths were conducted at 37 C inside an anaerobic chamber (Coy Laboratory Products, Grass Lake, MI). B. longum subsp. infantis EVC001 and NLS and B. breve 7051 were cultured for this experiment. To initiate experiments, single bacterial colonies from 48-hour growths were transferred to 3 ml of MRS (Difco BD, Franklin Lakes, NJ). After 18 hours of growth, bacterial cultures were standardized to an OD₆₀₀ of 1.0 and inoculated (10% w/vol) into 1.4 ml of RPMI 1640 (ThermoFisher Scientific, Waltham, MA) that lacked glucose and contained 25 mM HEPES and 2% (w/vol) of carbon source. The three carbon sources tested were galacto-oligosaccharides (GOS), 2′-fucosyllactose (2FL), and purified pooled human milk oligosaccharides (pooled HMO). Cultures were grown for 12 hours. Supernatant was harvested by centrifuging cells for 5 min at 16,000×g at 4 C. Supernatant was added to a new tube, flash frozen, and stored at −80 C until later processing.

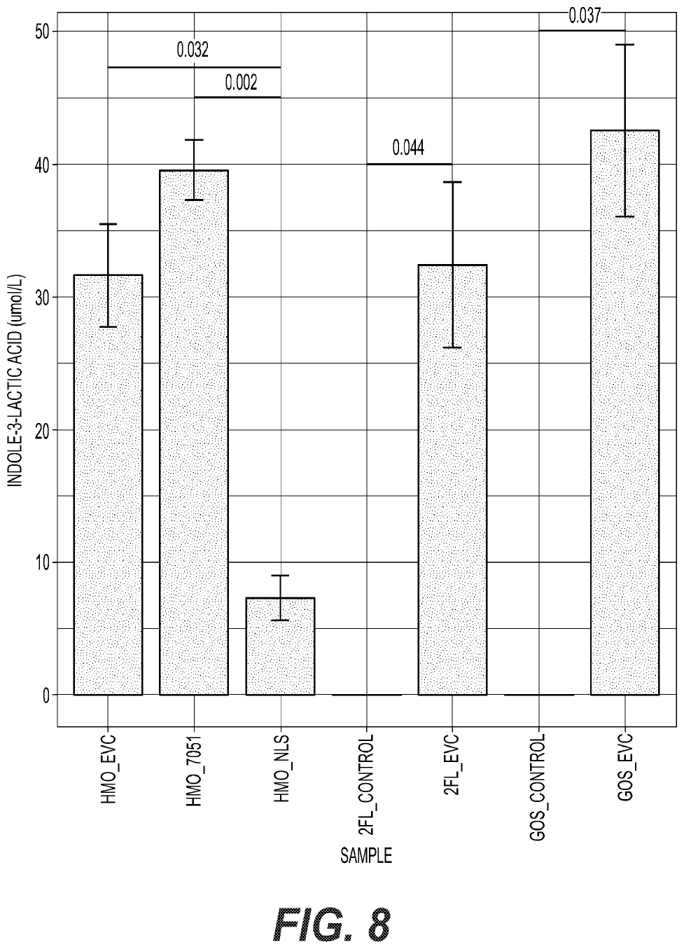

Indole-3-lactic acid production profile comparison. Three biological bacterial replicates and a single control lacking bacteria were analyzed per carbon source. Absolute abundance of indole-3-lactic acid was measured with Liquid Chromotography/Tandem Mass Spectrometry (LC-MS/MS) C18 Positive quantification on Thermo Vanquish Horizon uHPLC with Thermo Orbitrap Exploris instrument (ThermoFisher Scientific, Waltham, MA). Standards were prepared at 100 μM concentrations and diluted 1:2 to generate a 10-point standard curve. 500 ul of sample supernatant was used for LC-MS/MS processing using randomized sample injection. Data were converted for mzXML format and feature detection carried out using MZmine2. Features were annotated by searching against GNPS public and commercial NIST 2020 libraries. Comparison of absolute abundance between Control sample and bacterial culture samples was performed using the one sample t test. Pairwise comparisons were conducted between bacterial samples grown on pooled HMO. The Control sample for carbon source 2FL and GOS were used as the true value of the mean and was compared to the mean of 3 replicates, with the standard error also being computed. P values were corrected for false discovery. The results indicate that indole-3-lactic acid (FIG. 8) absolute abundance was distinct between Bifidobacterium species and strains depending on the carbon source. While EVC001 and NLS are the same bacterial subspecies, these two strains have distinct indole-3-lactic acid production on the pooled HMO. Alternatively, B. breve 7051 produced similar levels compared to EVC001. A purified individual HMO, 2FL, and GOS also resulted in high levels of indole-3-lactic acid. No inole-3-lactic acid was present in Control samples.

Insofar as the description above and the accompanying figures disclose any additional subject matter that is not within the scope of the claims below, the inventions are not dedicated to the public and the right to file one or more applications to claim such additional inventions is reserved.

### REFERENCES

Abrahamsson, T. R., Jakobsson, H. E., Andersson, A. F., Björkstén, B., Engstrand, L., and Jenmalm, M. C. (2014). Low gut microbiota diversity in early infancy precedes asthma at school age. Clin Exp Allergy 44, 842-850.
Abt, M. C., Osborne, L. C., Monticelli, L. A., Doering, T. A., Alenghat, T., Sonnenberg, G. F., Paley, M. A., Antenus, M., Williams, K. L., Erikson, J., et al. (2012). Commensal Bacteria Calibrate the Activation Threshold of Innate Antiviral Immunity. Immunity 37, 158-170.
Albert, K., Rani, A., and Sela, D. A. (2019). Comparative Pangenomics of the Mammalian Gut Commensal Bifidobacterium longum. Microorg 8, 7.
Apte, R. N., and Voronov, E. (2008). Is interleukin-1 a good or bad ‘guy’ in tumor immunobiology and immunotherapy? Immunol Rev 222, 222-241.
Arrieta, M.-C., Stiemsma, L. T., Dimitriu, P. A., Thorson, L., Russell, S., Yurist-Doutsch, S., Kuzeljevic, B., Gold, M. J., Britton, H. M., Lefebvre, D. L., et al. (2015). Early infancy microbial and metabolic alterations affect risk of childhood asthma. Science Translational Medicine 7, 307ra152-307ra152.
Arrieta, M.-C., Arévalo, A., Stiemsma, L., Dimitriu, P., Chico, M. E., Loor, S., Vaca, M., Boutin, R., Morien, E., Jin, M., et al. (2017). Associations between infant fungal and bacterial dysbiosis and childhood atopic wheeze in a nonindustrialized setting. Journal of Allergy and Clinical Immunology.
Avershina, E., Storrø, O., Øien, T., Johnsen, R., Pope, P., and Rudi, K. (2014). Major faecal microbiota shifts in composition and diversity with age in a geographically restricted cohort of mothers and their children. Fems Microbiol Ecol 87, 280-290.
Azad, M. B., Konya, T., Maughan, H., Guttman, D. S., Field, C. J., Chari, R. S., Sears, M. R., Becker, A. B., Scott, J. A., Kozyrskyj, A. L., et al. (2013). Gut microbiota of healthy Canadian infants: profiles by mode of delivery and infant diet at 4 months. Can Med Assoc J 185, 385-394.
Beghini, F., Mclver, L. J., Blanco-Miguez, A., Dubois, L., Asnicar, F., Maharjan, S., Mailyan, A., Thomas, A. M., Manghi, P., Valles-Colomer, M., et al. (2020). Integrating taxonomic, functional, and strain-level profiling of diverse microbial communities with bioBakery 3. Biorxiv 2020.11.19.388223.
Bokulich, N. A., Subramanian, S., Faith, J. J., Gevers, D., Gordon, J. I., Knight, R., Mills, D. A., and Caporaso, J. G. (2013). Quality-filtering vastly improves diversity estimates from Illumina amplicon sequencing. Nat Methods 10, 57-59.
Bolger, A. M., Lohse, M., and Usadel, B. (2014). Trimmomatic: a flexible trimmer for Illumina sequence data. Bioinformatics 30, 2114-2120.
Bray, N. L., Pimentel, H., Melsted, P., and Pachter, L. (2016). Near-optimal probabilistic RNA-seq quantification. Nat Biotechnol 34, 525-527.
Brodin, P., Duffy, D., and Quintana-Murci, L. (2019). A Call for Blood—In Human Immunology. Immunity 50, 1335-1336.
Butler, A., Hoffman, P., Smibert, P., Papalexi, E., and Satija, R. (2018). Integrating single-cell transcriptomic data across different conditions, technologies, and species. Nat Biotechnol 36, 411-420.
Byrnes, A. A., McArthur, J. C., and Karp, C. L. (2002). Interferon-therapy for multiple sclerosis induces reciprocal changes in interleukin-12 and interleukin-10 production. Ann Neurol 51, 165-174.
Call, L., Stoll, B., Oosterloo, B., Ajami, N., Sheikh, F., Wittke, A., Waworuntu, R., Berg, B., Petrosino, J., Olutoye, O., et al. (2018). Metabolomic signatures distinguish the impact of formula carbohydrates on disease outcome in a preterm piglet model of NEC. Microbiome 6, 111.
Cano-Gamez, E., Soskic, B., Roumeliotis, T. I., So, E., Smyth, D. J., Baldrighi, M., Willé, D., Nakic, N., Esparza-Gordillo, J., Larminie, C. G. C., et al. (2020). Single-cell transcriptomics identifies an effectorness gradient shaping the response of CD4+ T cells to cytokines. Nat Commun 17, 1801.
Caporaso, J. G., Kuczynski, J., Stombaugh, J., Bittinger, K., Bushman, F. D., Costello, E. K., Fierer, N., Peña, A. G., Goodrich, J. K., Gordon, J. I., et al. (2010). QIIME allows analysis of high-throughput community sequencing data. Nat Methods 7, 335-336.
Caporaso, J. G., Lauber, C. L., Walters, W. A., Berg-Lyons, D., Lozupone, C. A., Turnbaugh, P. J., Fierer, N., and Knight, R. (2011). Global patterns of 16S rRNA diversity at a depth of millions of sequences per sample. Proc National Acad Sci 108, 4516-4522.
Casaburi, G., and Frese, S. A. (2018). Colonization of breastfed infants by Bifidobacterium longum subsp. infantis EVC001 reduces virulence gene abundance. Hum Microbiome J 9, 7-10.
Casaburi, G., Duar, R. M., Vance, D. P., Mitchell, R., Contreras, L., Frese, S. A., Smilowitz, J. T., and Underwood, M. A. (2019). Early-life gut microbiome modulation reduces the abundance of antibiotic-resistant bacteria. Antimicrob Resist Infect Control 8, 131.
Casaburi, G., Duar, R. M., Brown, H., Mitchell, R. D., Kazi, S., Chew, S., Cagney, O., Flannery, R. L., Sylvester, K. G., Frese, S. A., et al. (2021). Metagenomic insights of the infant microbiome community structure and function across multiple sites in the United States. Sci Rep-Uk 11, 1472.
Chen, S., Zhou, Y., Chen, Y., and Gu, J. (2018). fastp: an ultra-fast all-in-one FASTQ preprocessor. Bioinformatics 34, 1884-1890.
Chen, Y., Tadepally, L., Mikes, J., and Brodin, P. (2020). Single-cell classification using learned cell phenotypes. Undefined.
Costea, P. I., Zeller, G., Sunagawa, S., Pelletier, E., Alberti, A., Levenez, F., Tramontano, M., Driessen, M., Hercog, R., Jung, F.-E., et al. (2017). Towards standards for human fecal sample processing in metagenomic studies. Nat Biotechnol 35, 1069-1076.
Czajkowski, M. D., Vance, D. P., Frese, S. A., and Casaburi, G. (2018). GenCoF: a graphical user interface to rapidly remove human genome contaminants from metagenomic datasets. Bioinformatics 35, 2318-2319.
Davis, M. M., and Brodin, P. (2018). Rebooting Human Immunology. Annu Rev Immunol 36, 1-22.
Dikopoulos, N., Bertoletti, A., Kröger, A., Hauser, H., Schirmbeck, R., and Reimann, J. (2005). Type I IFN Negatively Regulates CD8+ T Cell Responses through IL-10-Producing CD4+ T Regulatory 1 Cells. J Immunol 174, 99-109.
Dominguez-Bello, M., Godoy-Vitorino, F., Knight, R., and Blaser, M. J. (2019). Role of the microbiome in human development. Gut 68, 1108.
Duar, R. M., Henrick, B. M., Casaburi, G., and Frese, S. A. (2020a). Integrating the Ecosystem Services Framework to Define Dysbiosis of the Breastfed Infant Gut: The Role of B. infantis and Human Milk Oligosaccharides. Frontiers Nutrition 7, 33
Duar, R. M., Casaburi, G., Mitchell, R. D., Scofield, L. N. C., Ramirez, C. A. O., Barile, D., Henrick, B. M., and Frese, S. A. (2020b). Comparative Genome Analysis of Bifidobacterium longum subsp. infantis Strains Reveals Variation in Human Milk Oligosaccharide Utilization Genes among Commercial Probiotics. Nutrients 12, 3247.
Edgar, R. C. (2010). Search and clustering orders of magnitude faster than BLAST. Bioinformatics 26, 2460-2461.
Ehrlich, A. M., Henrick, B., Pacheco, A., Taft, D., Xu, G., Huda, N., Lozada-Contreras, M., Goodson, M., Slupsky, C., Mills, D., et al. (2018). Bifidobacterium grown on human milk oligosaccharides produce tryptophan metabolite Indole-3-lactic acid that significantly decreases inflammation in intestinal cells in vitro. Faseb J 32, 1b359-1b359.
Ehrlich, A. M., Pacheco, A. R., Henrick, B. M., Taft, D., Xu, G., Huda, M. N., Mishchuk, D., Goodson, M. L., Slupsky, C., Barile, D., et al. (2020). Indole-3-lactic acid associated with Bifidobacterium-dominated microbiota significantly decreases inflammation in intestinal epithelial cells. Bmc Microbiol 20, 357.
Franzosa, E. A., McIver, L. J., Rahnavard, G., Thompson, L. R., Schirmer, M., Weingart, G., Lipson, K. S., Knight, R., Caporaso, J. G., Segata, N., et al. (2018). Species-level functional profiling of metagenomes and metatranscriptomes. Nat Methods 15, 962-968.
Frese, S. A., Hutton, A. A., Contreras, L. N., Shaw, C. A., Palumbo, M. C., Casaburi, G., Xu, G., Davis, J. C. C., Lebrilla, C. B., Henrick, B. M., et al. (2017). Persistence of Supplemented Bifidobacterium longum subsp. infantis EVC001 in Breastfed Infants. Msphere 2, e00501-17.
Fukuda, S., Toh, H., Hase, K., Oshima, K., Nakanishi, Y., Yoshimura, K., Tobe, T., Clarke, J. M., Topping, D. L., Suzuki, T., et al. (2011). Bifidobacteria can protect from enteropathogenic infection through production of acetate. Nature 469, 543-547.
Gorfu, G., Rivera-Nieves, J., and Ley, K. (2009). Role of & #946; 7 Integrins in Intestinal Lymphocyte Homing and Retention. Curr Mol Med 9, 836-850.
Grzekowiak, ukasz, Collado, M. C., Mangani, C., Maleta, K., Laitinen, K., Ashorn, P., Isolauri, E., and Salminen, S. (2012). Distinct Gut Microbiota in Southeastern African and Northern European Infants. J Pediatr Gastr Nutr 54, 812-816.
Gu, J., Ni, X., Pan, X., Lu, H., Lu, Y., Zhao, J., Zheng, S. G., Hippen, K. L., Wang, X., and Lu, L. (2017). Human CD39hi regulatory T cells present stronger stability and function under inflammatory conditions. Cell Mol Immunol 14, 521-528.
Han, A., Newell, E. W., Glanville, J., Fernandez-Becker, N., Khosla, C., Chien, Y., and Davis, M. M. (2013). Dietary gluten triggers concomitant activation of CD4+ and CD8+ αβ T cells and γδ T cells in celiac disease. Proc National Acad Sci 110, 13073-13078.
Henrick, B. M., Chew, S., Casaburi, G., Brown, H. K., Frese, S. A., Zhou, Y., Underwood, M. A., and Smilowitz, J. T. (2019). Colonization by B. infantis EVC001 modulates enteric inflammation in exclusively breastfed infants. Pediatr Res 86, 749-757.
Huda, M. N., Lewis, Z., Kalanetra, K. M., Rashid, M., Ahmad, S. M., Raqib, R., Qadri, F., Underwood, M. A., Mills, D. A., and Stephensen, C. B. (2014). Stool Microbiota and Vaccine Responses of Infants. Pediatrics 134, e362-e372.
Hviid, A., Svanström, H., and Frisch, M. (2011). Antibiotic use and inflammatory bowel diseases in childhood. Gut 60, 49.
Ioannidis, M., Cerundolo, V., and Salio, M. (2020). The Immune Modulating Properties of Mucosal-Associated Invariant T Cells. Front Immunol/1, 1556.
Jacomy, M., Venturini, T., Heymann, S., and Bastian, M. (2014). ForceAtlas2, a continuous graph layout algorithm for handy network visualization designed for the Gephi software. PlosOne 9, e98679.
Jost, T., Lacroix, C., Braegger, C. P., and Chassard, C. (2012). New Insights in Gut Microbiota Establishment in Healthy Breast Fed Neonates. Plos One 7, e44595.
Knoop, K. A., Gustafsson, J. K., McDonald, K. G., Kulkarni, D. H., Coughlin, P. E., McCrate, S., Kim, D., Hsieh, C.-S., Hogan, S. P., Elson, C. O., et al. (2017). Microbial antigen encounter during a preweaning interval is critical for tolerance to gut bacteria. Science Immunology 2, eaao1314.
Laforest-Lapointe, I., and Arrieta, M.-C. (2017). Patterns of Early-Life Gut Microbial Colonization during Human Immune Development: An Ecological Perspective. Front Immunol 8, 788.
Laursen, M. F., Sakanaka, M., Burg, N. von, Andersen, D., Morbe, U., Rivollier, A., Pekmez, C. T., Moll, J. M., Michaelsen, K. F., Mølgaard, C., et al. (2020). Breastmilk-promoted bifidobacteria produce aromatic lactic acids in the infant gut. Biorxiv 2020.01.22.914994.
Lewis, Z. T., Totten, S. M., Smilowitz, J. T., Popovic, M., Parker, E., Lemay, D. G., Tassell, M. L. V., Miller, M. J., Jin, Y.-S., German, J. B., et al. (2015). Maternal fucosyltransferase 2 status affects the gut bifidobacterial communities of breastfed infants. Microbiome 3, 13.
Li, S., Rouphael, N., Duraisingham, S., Romero-Steiner, S., Presnell, S., Davis, C., Schmidt, D. S., Johnson, S. E., Milton, A., Rajam, G., et al. (2013). Molecular signatures of antibody responses derived from a systems biology study of five human vaccines. Nature Immunology/5, 195-204.
Lloyd-Price, J., Mahurkar, A., Rahnavard, G., Crabtree, J., Orvis, J., Hall, A. B., Brady, A., Creasy, H. H., McCracken, C., Giglio, M. G., et al. (2017). Strains, functions and dynamics in the expanded Human Microbiome Project. Nature 550, 61-66.
LoCascio, R. G., Ninonuevo, M. R., Freeman, S. L., Sela, D. A., Grimm, R., Lebrilla, C. B., Mills, D. A., and German, J. B. (2007). Glycoprofiling of Bifidobacterial Consumption of Human Milk Oligosaccharides Demonstrates Strain Specific, Preferential Consumption of Small Chain Glycans Secreted in Early Human Lactation. J Agr Food Chem 55, 8914-8919.
LoCascio, R. G., Desai, P., Sela, D. A., Weimer, B., and Mills, D. A. (2010). Broad Conservation of Milk Utilization Genes in Bifidobacterium longum subsp. infantis as Revealed by Comparative Genomic Hybridization ∇ †. Appl Environ Microb 76, 7373-7381.
Love, M. I., Huber, W., and Anders, S. (2014). Moderated estimation of fold change and dispersion for RNA-seq data with DESeq2. Genome Biol 15, 550.
Lundberg, M., Eriksson, A., Tran, B., Assarsson, E., and Fredriksson, S. (2011). Homogeneous antibody-based proximity extension assays provide sensitive and specific detection of low-abundant proteins in human blood. Nucleic Acids Res 39, e102-e102.
Maggi, L., Santarlasci, V., Capone, M., Peired, A., Frosali, F., Crome, S. Q., Querci, V., Fambrini, M., Liotta, F., Levings, M. K., et al. (2010). CD161 is a marker of all human IL-17-producing T-cell subsets and is induced by RORC. Eur J Immunol 40, 2174-2181.
Mair, F., Erickson, J. R., Voillet, V., Simoni, Y., Bi, T., Tyznik, A. J., Martin, J., Gottardo, R., Newell, E. W., and Prlic, M. (2020). A Targeted Multi-omic Analysis Approach Measures Protein Expression and Low-Abundance Transcripts on the Single-Cell Level. Cell Reports 31, 107499. Meng, D., Sommella, E., Salviati, E., Campiglia, P., Ganguli, K., Djebali, K., Zhu, W., and Walker, W. A. (2020). Indole-3-lactic acid, a metabolite of tryptophan, secreted by Bifidobacterium longum subspecies infantis is anti-inflammatory in the immature intestine. Pediatr Res 88, 209-217.
Mikes, J., Olin, A., Lakshmikanth, T., Chen, Y., and Brodin, P. (2019). Mass Cytometry, Methods and Protocols. Methods Mol Biology Clifton N J 1989, 111-123.
Mohammadkhah, A. I., Simpson, E. B., Patterson, S. G., and Ferguson, J. F. (2018). Development of the Gut Microbiome in Children, and Lifetime Implications for Obesity and Cardiometabolic Disease. Children 5, 160.
Nabhani, Z., Dulauroy, S., Marques, R., Cousu, C., Bounny, S., Déjardin, F., Sparwasser, T., Bérard, M., Cerf-Bensussan, N., and Eberl, G. (2019). A Weaning Reaction to Microbiota Is Required for Resistance to Immunopathologies in the Adult. Immunity.
Narasimhan, P. B., Marcovecchio, P., Hamers, A. A. J., and Hedrick, C. C. (2019). Nonclassical Monocytes in Health and Disease. Annu Rev Immunol 37, 439-456.
Nguyen, M., Holdbrooks, H., Mishra, P., Abrantes, M. A., Eskew, S., Garma, M., Oca, C.-G., McGuckin, C., Hein, C. B., Mitchell, R. D., et al. (2021). Impact of Probiotic B. infantis EVC001 Feeding in Premature Infants on the Gut Microbiome, Nosocomially Acquired Antibiotic Resistance, and Enteric Inflammation. Frontiers Pediatrics 9, 618009.
Olin, A., Henckel, E., Chen, Y., Lakshmikanth, T., Pou, C., Mikes, J., Gustafsson, A., Bernhardsson, A. K., Zhang, C., Bohlin, K., et al. (2018). Stereotypic Immune System Development in Newborn Children. Cell 174, 1277-1292.e14.
Pou, C., Nkulikiyimfura, D., Henckel, E., Olin, A., Lakshmikanth, T., Mikes, J., Wang, J., Chen, Y., Bernhardsson, A., Gustafsson, A., et al. (2019). The repertoire of maternal anti-viral antibodies in human newborns. Nature Medicine 1-6.
Pré, F. M. du, Berkel, L. A. van, Raki, M., Leeuwen, M. A. van, Ruiter, L. F. de, Broere, F., Borg, M. N. D. ter, Lund, F. E., Escher, J. C., Lundin, K. E. A., et al. (2011). CD62LnegCD38+ Expression on Circulating CD4+ T Cells Identifies Mucosally Differentiated Cells in Protein Fed Mice and in Human Celiac Disease Patients and Controls. Am J Gastroenterol 106, 1147-1159.
Renz, H., and Skevaki, C. (2020). Early life microbial exposures and allergy risks: opportunities for prevention. Nat Rev Immunol 1-15.
Rhoads, J. M., Collins, J., Fatheree, N. Y., Hashmi, S. S., Taylor, C. M., Luo, M., Hoang, T. K., Gleason, W. A., Arsdall, M. R. V., Navarro, F., et al. (2018). Infant Colic Represents Gut Inflammation and Dysbiosis. J Pediatrics 203, 55-61.e3.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47-e47.
Rodriguez, L., Pekkarinen, P. T., Lakshmikanth, T., Tan, Z., Consiglio, C. R., Pou, C., Chen, Y., Mugabo, C. H., Nguyen, N. A., Nowlan, K., et al. (2020). Systems-level immunomonitoring from acute to recovery phase of severe COVID-19. Cell Reports Medicine 100078.
Roos, S., Dicksved, J., Tarasco, V., Locatelli, E., Ricceri, F., Grandin, U., and Savino, F. (2013). 454 Pyrosequencing Analysis on Faecal Samples from a Randomized DBPC Trial of Colicky Infants Treated with Lactobacillus reuteri DSM 17938. Plos One 8, e56710.
Sandin, A., Bråbäck, L., Norin, E., and Björkstén, B. (2009). Faecal short chain fatty acid pattern and allergy in early childhood. Acta Paediatr 98, 823-827.
Schaupp, L., Muth, S., Rogell, L., Kofoed-Branzk, M., Melchior, F., Lienenklaus, S., Ganal-Vonarburg, S. C., Klein, M., Guendel, F., Hain, T., et al. (2020). Microbiota-Induced Type I Interferons Instruct a Poised Basal State of Dendritic Cells. Cell 181, 1080-1096.e19.
Scholz, M., Ward, D. V., Pasolli, E., Tolio, T., Zolfo, M., Asnicar, F., Truong, D. T., Tett, A., Morrow, A. L., and Segata, N. (2016). Strain-level microbial epidemiology and population genomics from shotgun metagenomics. Nat Methods 13, 435-438.
Sela, D. A. (2011). Bifidobacterial utilization of human milk oligosaccharides. Int J Food Microbiol 149, 58-64.
Sela, D. A., and Mills, D. A. (2010). Nursing our microbiota: molecular linkages between bifidobacteria and milk oligosaccharides. Trends Microbiol 18, 298-307.
Sela, D. A., Chapman, J., Adeuya, A., Kim, J. H., Chen, F., Whitehead, T. R., Lapidus, A., Rokhsar, D. S., Lebrilla, C. B., German, J. B., et al. (2008). The genome sequence of Bifidobacterium longum subsp. infantis reveals adaptations for milk utilization within the infant microbiome. Proc National Acad Sci 105, 18964-18969.
Shin, N.-R., Whon, T. W., and Bae, J.-W. (2015). Proteobacteria: microbial signature of dysbiosis in gut microbiota. Trends Biotechnol 33, 496-503.
Smilowitz, J. T., Moya, J., Breck, M. A., Cook, C., Fineberg, A., Angkustsiri, K., and Underwood, M. A. (2017). Safety and tolerability of Bifidobacterium longum subspecies infantis EVC001 supplementation in healthy term breastfed infants: a phase I clinical trial. Bmc Pediatr 17, 133.
Smith, P. M., Howitt, M. R., Panikov, N., Michaud, M., Gallini, C. A., Bohlooly-Y, M., Glickman, J. N., and Garrett, W. S. (2013). The Microbial Metabolites, Short-Chain Fatty Acids, Regulate Colonic Treg Cell Homeostasis. Science 341, 569-573.
Sonnenburg, E. D., and Sonnenburg, J. L. (2019). The ancestral and industrialized gut microbiota and implications for human health. Nature Reviews Microbiology 17, 383-390.
Stefan, K. L., Kim, M. V., Iwasaki, A., and Kasper, D. L. (2020). Commensal Microbiota Modulation of Natural Resistance to Virus Infection. Cell 183, 1312-1324.e10.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: A knowledge-based approach for interpreting genome-wide expression profiles. P Natl Acad Sci Usa 102, 15545-15550.
Sundblad, V., Quintar, A. A., Morosi, L. G., Niveloni, S. I., Cabanne, A., Smecuol, E., Mauriño, E., Mariño, K. V., Bai, J. C., Maldonado, C. A., et al. (2018). Galectins in Intestinal Inflammation: Galectin-1 Expression Delineates Response to Treatment in Celiac Disease Patients. Front Immunol 9, 379.
Thome, J. J. C., Bickham, K. L., Ohmura, Y., Kubota, M., Matsuoka, N., Gordon, C., Granot, T., Griesemer, A., Lerner, H., Kato, T., et al. (2016). Early-life compartmentalization of human T cell differentiation and regulatory function in mucosal and lymphoid tissues. Nat Med 22, 72-77.
Traag, V. A., Waltman, L., and Eck, N. J. van (2019). From Louvain to Leiden: guaranteeing well-connected communities. Sci Rep-Uk 9, 5233.
Trompette, A., Gollwitzer, E. S., Yadava, K., Sichelstiel, A. K., Sprenger, N., Ngom-Bru, C., Blanchard, C., Junt, T., Nicod, L. P., Harris, N. L., et al. (2014). Gut microbiota metabolism of dietary fiber influences allergic airway disease and hematopoiesis. Nat Med 20, 159-166.
Truong, D. T., Franzosa, E. A., Tickle, T. L., Scholz, M., Weingart, G., Pasolli, E., Tett, A., Huttenhower, C., and Segata, N. (2015). MetaPhlAn2 for enhanced metagenomic taxonomic profiling. Nat Methods 12, 902-903.
Uhlen, M., Karlsson, M. J., Zhong, W., Tebani, A., Pou, C., Mikes, J., Lakshmikanth, T., Forsström, B., Edfors, F., Odeberg, J., et al. (2019). A genome-wide transcriptomic analysis of protein-coding genes in human blood cells. Sci New York N Y 366.
Underwood, M. A., German, J. B., Lebrilla, C. B., and Mills, D. A. (2015). Bifidobacterium longum subspecies infantis: champion colonizer of the infant gut. Pediatr Res 77, 229-235.
Vatanen, T., Kostic, A. D., d'Hennezel, E., Siljander, H., Franzosa, E. A., Yassour, M., Kolde, R., Vlamakis, H., Arthur, T. D., Hämäläinen, A.-M., et al. (2016). Variation in Microbiome LPS Immunogenicity Contributes to Autoimmunity in Humans. Cell 165, 842-853.
Walters, W., Hyde, E. R., Berg-Lyons, D., Ackermann, G., Humphrey, G., Parada, A., Gilbert, J. A., Jansson, J. K., Caporaso, J. G., Fuhrman, J. A., et al. (2016). Improved Bacterial 16S rRNA Gene (V4 and V4-5) and Fungal Internal Transcribed Spacer Marker Gene Primers for Microbial Community Surveys. Msystems I, e00009-15.
Wolf, F. A., Hamey, F. K., Plass, M., Solana, J., Dahlin, J. S., Göttgens, B., Rajewsky, N., Simon, L., and Theis, F. J. (2019). PAGA: graph abstraction reconciles clustering with trajectory inference through a topology preserving map of single cells. Genome Biol 20, 59.
Wood, D. E., Lu, J., and Langmead, B. (2019). Improved metagenomic analysis with Kraken 2. Genome Biol 20, 257.
Yaseen, H., Butenko, S., Polishuk-Zotkin, I., Schif-Zuck, S., Pérez-Sáez, J. M., Rabinovich, G. A., and Ariel, A. (2020). Galectin-1 Facilitates Macrophage Reprogramming and Resolution of Inflammation Through IFN-β. Front Pharmacol 11, 901.
Yoshida, H., and Hunter, C. A. (2015). The Immunobiology of Interleukin-27. Annu Rev Immunol 33, 417-443.

1. A T cell polarizing composition comprising a plurality of metabolites selected from metabolites enriched in non-dysbiotic feces, preferably including one or more of acetate, lactate, indole 3 lactate, 3-(4-hydroxyphenyl)lactate, or phenyllactate, bile acids (cholate, chenodeoxycholate, cholate sulfate), or IFNβ.

2. The T cell polarizing composition of claim 1, wherein the metabolites comprise at least 2 of ILA (indole lactate), lactate, acetate, TGFβ, IL-2, IL-10, and/or IL-27.

3. The T cell polarizing composition of claim 1 or 2, wherein the T cell polarizing composition also comprises at least one cytokine, preferably the cytokine is IFNβ, IL-2, IL-22, Il-27, TGFβ and/or IFNγ an interferon, more preferably IFNβ.

4. The T cell polarizing composition of any one of claims 1-3, wherein the T cell polarizing composition is a sterile fecal water, culture supernatant from bacterial culture, preferably wherein the bacterial culture comprises B. infantis.

5. The T cell polarizing composition of any one of claim 1-4, wherein the T cell polarizing composition is dried, preferably powdered.

6. A method of producing the T cell polarizing composition of any one of claim 1-4, comprising culturing Bifidobacterium sp. cells in the presence of mammalian milk oligosaccharides (MMO) and/or GOS.

7. The method of claim 6, wherein the MMO comprises one or more of LNT, LnNT, 3SL, 6SL, 2FL, 3FL, BMO and/or BCO.

8. The method of claim 6 or claim 7, whereby indole lactic acid (ILA) accumulates in the culture.

9. A method of producing the T cell polarizing composition of any one of claims 6-8, comprising mixing interferon beta (IFNβ), acetate, lactate, and/or ILA.

10. The method of claim any one of claim 6-9, wherein said polarizing composition was synthesized to mimic certain features of the metabolic profile of feces from a non-dysbiotic infant comprising cells or the metabolites generated by Bifidobacterium sp.

11. The method of any one of claims 6-10, wherein said Bifidobacterium sp. of said polarizing composition includes one or more species selected from B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. pseudocatanulatum, and/or B. pseudolongum.

12. The method of claim 11 wherein the Bifidobacterium species is B. longum subsp. infantis (B. infantis).

13. The method of claim 12, wherein the B. infantis is the strain EVC001 or genetically equivalent to B. infantis EVC001.

14. The method of claim 2, wherein said polarizing composition comprises cells of Lactobacillus sp.

15. The method of claim 7 wherein said Lactobacillus sp. is selected from L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius L. paracasei, L. kisonensis, L. paralimentarius, L. perolens, L. apis, L. ghanensis, L. dextrinicus, L. shenzenensis, and/or L. harbinensis.

16. The method of any one of claims 6-15, wherein said polarizing composition comprises indole lactate 3 (ILA).

17. The method of any one of claims 6-16, wherein said polarizing composition comprises interferon beta.

18. The method of any one of claims 6-17, wherein the polarizing composition comprises cell-free fecal water.

19. The method of claim 18, wherein the fecal water is sterile.

20. A T cell preparation comprising:
a. cultured human T regulatory and helper T cells polarized from a purified naïve T cell population; and
b. an activity profile comprising desired frequency or ratio of T regulatory and T helper cell types.

21. A T cell preparation of claim 20, wherein the desired activity profile comprises a frequency of less than 1%, less than 5%, less than 10% residual naïve T cells.

22. A T cell preparation of any one of claims 20-21, wherein purified naïve T cells express CD3+CD4+CD45RA+ and/or are negative for CD8, CD14, CD19, or CD56.

23. A T cell preparation of any one of claims 20-22, wherein the frequency of at least 1, 2, 5, 10, 20, 40, or 80% of the total CD4+ T cell population in the T cell preparation are regulatory T cells expressing FOXP3+.

24. A T cell preparation of any one of claims 20-23, wherein the frequency of at least 10%, 15%, 25%, 30%, 40%, 50%, 60%, 70%, and 80% of the total CD4+ T cell population in the T cell preparation are T Helper Type I (Th1) cells.

25. A T cell preparation of any one of claims 20-24, wherein the frequency of less than 50%, 40%, 30%, 20%, 10%, 5%, 1% of the T cells in the T cell preparation are T Helper Type 2 cells (Th2).

26. A T cell preparation of any one of claims 20-25, wherein the frequency of less than 50%, 40%, 30%, 20%, 10%, 5%, 1% of the T cells in the T cell preparation are T helper type 17 (Th17) cells.

27. A T cell preparation of any one of claims 20-26, wherein negative regulators are upregulated in Th0, Th2, and Th17 but not Th1 CD4+ T cells.

28. A T cell preparation of claim 27, wherein the negative regulator is galectin-1, and galectin-1 expression is upregulated in T cell preparation compared to naïve T cells.

29. A T cell preparation of any one of claims 20-28, wherein Interferon beta is expressed.

30. A T cell preparation of any one of claims 20-29, wherein the ratio of FoxP3+CD4+ T cells to Th2 or Th17 CD4+ T cells are greater than 1:1, 2:1, 4:1, 8:1, 16:1, 32:1, 64:1, 128:1, 256:1, 512:1, 1024:1.

31. A T cell preparation of any one of claims 20-30, wherein the ratio of Th1 to Th2 CD4+ T cells are greater than 1:1, 2:1, 4:1, 8:1, 16:1, 32:1, 64:1, 128:1.

32. A T cell preparation of any one of claims 20-31, where the cultured human T regulatory and helper T cells are at least 10 cultured cells per unit dose, at least 100, at least 1000, at least 10,000, at least 100,000, at least 1 million or at least 1 billion cultured cells per unit dose, or preferably 10¹-10⁹ cells/ml.

33. A method of polarizing T cells, comprising culturing stem cells obtained from a subject under conditions whereby at least some of the cells differentiate into T cells, and wherein a polarizing composition from any one of claims 1-5 is applied to the T cells, and polarized T cells are recovered after polarization.

34. A method wherein any polarizing composition from any one of claims 1-5 is applied to a population of Naïve T cells separated from other T cells.

35. A method according to any one of claims 33-34, wherein any polarizing composition from any one of claims 1-5 is applied to a mixed population of T cells separated from other white blood cells.

36. The method of any one of claims 33-35, wherein T cells are recovered after polarization.

37. A method of making a T cell preparation, wherein the composition is made ex vivo, said method of making T cell preparation comprising
a. collecting blood;
b. isolating naïve T cells or enriching naïve T cells;
c. exposing naïve T cells to a polarizing composition;
d. recovering polarized T cells; and
e. optionally administering a composition of the T cell preparation to a subject in need thereof.

38. The method of any one of claims 33-37, wherein the polarizing composition comprises indole lactic acid (ILA).

39. The method of any one of claims 33-38, wherein the polarizing composition is a sterile water extract of feces obtained from a non-dysbiotic infant (i.e., fecal water), an artificial stool, or purified metabolites and/or cytokines formulated to mimic gut signals.

40. The method of claim 39, wherein the feces for the sterile water extract of feces is selected based on one or more of the following criteria: i) feces containing more than 20% Bifidobacterium sp, more than 30% Bifidobacterium sp., more than 40% Bifidobacterium sp, more than 50% more than Bifidobacterium sp, more than 60% Bifidobacterium sp., 70% Bifidobacterium sp., 80% Bifidobacterium sp., or more than 90% Bifidobacterium sp. by relative abundance in the non-dysbiotic infant microbiome; ii) feces containing B. infantis as more than 20%, more than 30%, more than 40%, more than 50%, more than 60%, more than 70%, more than 80%, or more than 90% by relative abundance in a non-dysbiotic infant microbiome; iii) feces containing B. infantis genetically and/or functionally equivalent to certain features of B. infantis strain EVC001.

41. The method of claim 40, wherein the certain features of B. infantis are selected from the ability to consume certain types of HMO and/or the release of N-glycans through increased expression of cell surface markers, optionally wherein the cell surface markers comprise endo-BI-1.

42. The method of any one of claims 39-41, wherein said feces comprises bacterial cells which are less than 40% Enterobacteriaceae, less than 30% Enterobacteriaceae, less than 20% Enterobacteriaceae, less than 10% Enterobacteriaceae, less than 5% Enterobacteriaceae, less than 2% Enterobacteriaceae, or less than 1% Enterobacteriaceae by relative abundance in the non-dysbiotic infant microbiome.

43. The method of any one of claims 33-42, wherein the recovered polarized T cells are administered to a subject in need thereof.

44. The method of claim 43, wherein the subject is the source of the naïve T cells.

45. The method of claim 44, wherein the naïve T cells are obtained from the subject.

46. The method of any one of claims 44-45, wherein the recovered polarized T cells are reintroduced into the same subject.

47. A method of enhancing immune expansion and/or reducing intestinal inflammation in a human subject in need, comprising administering the T cell polarizing composition or T cell preparation of any one of the preceding claims to said subject.

48. The method of administering the T cell preparation of any one of claims 20-32 to a subject in need wherein the subject in need is an infant less than 1 week, less than three (3) months chronological age, optionally the infant is preterm.

49. The method of administering the T cell preparation of any one of claims 20-32 to a subject in need, wherein the subject is an infant more than three (3) months chronological age.

50. The method of administering the T cell preparation of any one of claims 20-32 to a subject in need, wherein the subject is 1-3 years, 4-11 years old, 12-15 years, 16-30 years, more than 30 years, more than 40 years, more than 50 years, more than 60 years, more than 70 years, more than 80 years

51. The method of any one of claims 43-50, wherein T cell the polarizing composition or T cell preparation is administered intravenously.

52. The method of any one of claims 43-50, wherein the T cell polarizing composition or T cell preparation is administered orally.

53. The method of any one of claims 43-50, wherein the T cell polarizing composition or T cell preparation is administered by enema or suppository.

54. The method of any one of claims 43-53, wherein a subject in need of the treatment has an autoimmune disease selected from the group consisting of MS, IBD (Crohn's, ulcerative colitis), Celiac's disease, type I diabetes, atopic wheeze, and atopic dermatitis.

55. A method of any one of claims 43-55, wherein the patient is has a chronic viral infection.

56. The method of claim 55 wherein the viral infection is selected from HIV or Hepatitis A, B, or C or herpes simplex.

57. A method of any one of claims 43-53, wherein the subject has cancer.

58. The method of claim 57 wherein the cancer is colon cancer, leukemia, or pancreatic cancer.

59. A method of any one of claims 43-53, wherein the subject is at risk for, or has an autoimmune disease.

60. A method of treating a subject in need of increasing circulating regulatory T cells, comprising delivering a polarizing composition according to any one of claims 1-19 orally or rectally.

61. A method of 60, wherein the polarizing composition further comprises microbiome modulators delivered to the gut, wherein microbiome modulators are selected from a prebiotic, probiotic and/or post-biotic preparations.

62. A method of treating a subject in need of increasing circulating regulatory T cells comprising administering the polarizing composition of any one of claims 1-19, wherein the administered polarizing composition increases total circulating regulatory T cells to 5-10% of all CD4+ T cells.

63. A T cell preparation of any one of claims 20-32, wherein the T cell preparation is capable of inhibiting Th17 cell expansion in vivo by at least 10%, at least 30%, or at 50% relative to pre-administration levels.

66. A method of increasing and/or maintaining a level of circulating T cell population in a mammal, comprising:
a. selecting a mammal having insufficient Regulatory T cells;
b. collecting and isolating naïve T cell;
c. applying a composition to polarize naïve T cells to Th1 and T regulatory cells; and
d. administering to the mammal the T cell preparation.

67. The method of claim 66, further comprising:
e. monitoring the level of T cell populations of said mammal and continuing steps (b) and/or (c) to maintain sufficient T cell levels; and
f. optionally adding microbiome modulators and polarizing compositions as oral or rectal to further support circulating T cell populations.
